SEQUENCE LISTING (74 sequences)

SEQ ID NO: 1 - protein, 17 aa, Artificial Sequence
  synthetic peptide
LVPRGSPGISGGGGGIR

SEQ ID NO: 2 - protein, 70 aa, Mus musculus
ESGKRMDCPALPPGWKKEEVIRKSGLSAGKSDVYYFSPSGKKFRSKPQLARYLGNAVDLSSFDFRTGKMM

SEQ ID NO: 3 - DNA, 16 bp, Artificial Sequence
  synthetic oligonucleotide primer
gcgggaccctccagaa

SEQ ID NO: 4 - DNA, 20 bp, Artificial Sequence
  synthetic oligonucletide primer
actcactggtggcgaagact

SEQ ID NO: 5 - DNA, 25 bp, Artificial Sequence
  synthetic oligonucleotide probe
accacccttataaggctcggaggcc

SEQ ID NO: 6 - DNA, 20 bp, Artificial Sequence
  synthetic oligonucleotide primer
ggtacgaaaaggcggaaaga

SEQ ID NO: 7 - DNA, 20 bp, Artificial Sequence
  synthetic oligonucleotide primer
tgtgggaaagctggaatatc

SEQ ID NO: 8 - DNA, 45 bp, Artificial Sequence
  synthetic oligonucleotide primer
cacaggtcaaaggtcataaaaatgatttcccataccaagcaccgt

SEQ ID NO: 9 - DNA, 17 bp, Artificial Sequence
  synthetic oligonucleotide primer
gtcctcctcacactccg

SEQ ID NO: 10 - DNA, 34 bp, Artificial Sequence
  synthetic oligonucleotide primer
caccgtcgaatctctccaccgtgtgcccaagacc

SEQ ID NO: 11 - DNA, 22 bp, Artificial Sequence
  thetic oligonucleotide primer
gtgcctttcattccatccagcc

SEQ ID NO: 12 - DNA, 35 bp, Artificial Sequence
  synthetic oligonucleotide primer
ccaagaagccacacgacaagcgggaccctccagaa

SEQ ID NO: 13 - DNA, 20 bp, Artificial Sequence
  synthetic oligonucleotide primer
actcactggtggcgaagact

SEQ ID NO: 14 - DNA, 34 bp, Artificial Sequence
  synthetic oligonucleotide primer
cctccatcccaaagtaacctctgctccctccgaa

SEQ ID NO: 15 - DNA, 18 bp, Artificial Sequence
  synthetic oligonucleotide primer
ccgatggcctagacactg

SEQ ID NO: 16 - DNA, 42 bp, Artificial Sequence
  synthetic oligonucleotide primer
gaaaggactacaaaggacagaaggtacgaaaaggcggaaaga

SEQ ID NO: 17 - DNA, 20 bp, Artificial Sequence
  synthetic oligonucleotide primer
tgtgggaaagctggaatatc

SEQ ID NO: 18 - DNA, 43 bp, Artificial Sequence
  synthetic oligonucleotide primer
cgaaaatgcatctgagtagcaacctccgcctaaaatccctatg

SEQ ID NO: 19 - DNA, 19 bp, Artificial Sequence
  synthetic oligonucleotide primer
ggtatcctgtccgctcgca

SEQ ID NO: 20 - DNA, 89 bp, Artificial Sequence
  synthetic oligonucleotide
catttttatgacctttgacctgtgaaatttatgtttgacagatcttacaatgcatgctataataccactaacggtgctttaaaattccg

SEQ ID NO: 21 - DNA, 65 bp, Artificial Sequence
  synthetic oligonucleotide
cggaattttaaagcaccgttagtggtattatagcatgcattgtaagatctgtcaaacataaattt

SEQ ID NO: 22 - DNA, 82 bp, Artificial Sequence
  synthetic oligonucleotide
gtcgtgtggcttcttggaaatttatgtttgacagatcttacaatgcatgctataataccactgaaggtgatataaaattccg

SEQ ID NO: 23 - DNA, 65 bp, Artificial Sequence
  synthetic oligonucleotide
cggaattttatatcaccttcagtggtattatagcatgcattgtaagatctgtcaaacataaattt

SEQ ID NO: 24 - DNA, 64 bp, Artificial Sequence
  synthetic oligonucleotide
tactttgggatggagggctgctggaggcgggtataatttagccagcaccgaatagttacggtcg

SEQ ID NO: 25 - DNA, 48 bp, Artificial Sequence
  synthetic oligonucleotide
cgaccgtaactattcggtgcttagggcagcgcccccgcctccacgagc

SEQ ID NO: 26 - DNA, 85 bp, Artificial Sequence
  synthetic oligonucleotide
catttttatgacctttgacctgtggctgttgacacagaataaacgctcaatgtacaatgggatggagaggtgctttagtagtgtt

SEQ ID NO: 27 - DNA, 77 bp, Artificial Sequence
  synthetic oligonucleotide
ggagagattcgacggtgctgttgacacagaataaacgctcaatgtacaatgggatggagaggtgctttagtagtgtt

SEQ ID NO: 28 - DNA, 78 bp, Artificial Sequence
  synthetic oligonucleotide
gtcgtgtggcttcttgggctgttgacacagaataaacgctcaatgtacaatgggatggagaggtgctttagtagtgtt

SEQ ID NO: 29 - DNA, 77 bp, Artificial Sequence
  synthetic oligonucleotide
tactttgggatggagggctgttgacacagaataaacgctcaatgtacaatgggatggagaggtgctttagtagtgtt

SEQ ID NO: 30 - DNA, 83 bp, Artificial Sequence
  synthetic oligonucleotide
tctgtcctttgtagtcctttcggctgttgacacagaataaacgctcaatgtacaatgggatggagaggtgctttagtagtgtt

SEQ ID NO: 31 - DNA, 81 bp, Artificial Sequence
  synthetic oligonucleotide
gctactcagatgcattttcggctgttgacacagaataaacgctcaatgtacaatgggatggagaggtgctttagtagtgtt

SEQ ID NO: 32 - DNA, 61 bp, Artificial Sequence
  synthetic oligonucleotide
aacactactaaagcacctctccatcccattgtacattgagcgtttattctgtgtcaacagc

SEQ ID NO: 33 - DNA, 60 bp, Artificial Sequence
  synthetic oligonucleotide primer
agagaattttttcataaacattaaatgtacaatgggaacgagaaccgtgtgcccaagacc

SEQ ID NO: 34 - DNA, 17 bp, Artificial Sequence
  synthetic oligonucleotide primer
ctgcctagggagagaga

SEQ ID NO: 35 - DNA, 63 bp, Artificial Sequence
  synthetic oligonucleotide primer
gctgttgacaattaataaacgctcaatgtacaatgggactgagactcttaggcttctggtggc

SEQ ID NO: 36 - DNA, 17 bp, Artificial Sequence
  synthetic oligonucleotide primer
cctgtggtgggcgatgc

SEQ ID NO: 37 - DNA, 65 bp, Artificial Sequence
  synthetic oligonucleotide primer
tgcgaaccttgactataaaaattcaatgtacaatgggacggagaaggtacgaaaaggcggaaaga

SEQ ID NO: 38 - DNA, 20 bp, Artificial Sequence
  synthetic oligonucleotide primer
tgtgggaaagctggaatatc

SEQ ID NO: 39 - DNA, 65 bp, Artificial Sequence
  synthetic oligonucleotide primer
gctgttgacacagttcaaacgctcaatgtaaaatgggacaatcacctccgcctaaaatccctatg

SEQ ID NO: 40 - DNA, 20 bp, Artificial Sequence
  synthetic oligonucleotide primer
cttgctgttgtgccgttctg

SEQ ID NO: 41 - DNA, 60 bp, Artificial Sequence
  synthetic oligonucleotide primer
gctgaagacacagaataaacgatcaatgtataatgggactgagagcgggaccctccagaa

SEQ ID NO: 42 - DNA, 44 bp, Artificial Sequence
  synthetic oligonucleotide primer
caggtaaagatccagaacccaacatgctcggagttaatagcacc

SEQ ID NO: 43 - DNA, 19 bp, Artificial Sequence
  synthetic oligonucleotide primer
gcgctacctgattcaattc

SEQ ID NO: 44 - DNA, 1101 bp, Homo sapiens
ttaaaaaaatctgttttgttctatgtgattttcccataccaagcaccgtgcccggcacaagctgggatcccagtacacatctcgggacggaagaaccgtgtttccctagaacccagtcagagggcagcttagcaatgtgtcacaggtggggcgcccgcgttccgggcggacgcactggctccccggccggcgtgggtgtggggcgagtgggtgtgtgcggggtgtgcgcggtagagcgcgccagcgagcccggagcgcggagctgggaggagcagcgagcgccgcgcagaacccgcagcgccggcctggcagggcagctcggaggtgggtgggccgcgccgccagcccgcttgcagggtccccattggccgcctgccggccgccctccgcccaaaaggcggcaaggagccgagaggctgcttcggagtgtgaggaggacagccggaccgagccaacgccggggactttgttccctccgcggaggggactcggcaactcgcagcggcagggtctggggccggcgcctgggagggatctgcgccccccactcactccctagctgtgttcccgccgccgccccggctagtctccggcgctggcgcctatggtcggcctccgacagcgctccggagggaccgggggagctcccaggcgcccgggactggagactgatgcatgagggggctacggaggcgcaggagcggtggtgatggtctgggaagcggagctgaagtgccctgggctttggtgaggcgtgacagtttatcatgaccgtgttcaggcaggaaaacgtggatgattactacgacaccggcgaggaacttggcagtggacagtttgcggttgtgaagaaatgccgtgagaaaagcaccggcctccagtatgccgccaaattcatcaagaaaaggaggactaagtccagccggcggggtgtgagccgcgaggacatcgagcgggaggtcagcatcctgaaggagatccagcaccccaatgtcatcaccctgcacgaggtctatgagaacaagacggacgtcatcctgatcttggaactcgttgcaggtggcgagctgtttgacttcttagctgaaaaggaatctttaa

SEQ ID NO: 45 - DNA, 2666 bp, Homo sapiens
ttaagcaaagattatcaccaggcaggctaaacttagcaaccggcttttagctagaagggcagggggctggtgtcaggttatgctgggccagcaaagaggcccgggatccccctcccatgcacctgctgatgggccaaggccaccccaccccacccccttccttacaagtgttcagcaccctcccatcccacactcacaaacctggccctctgccctcctaccagaagaatggatcccctgtgggagggggcaggggacctgttcccaccgtgtgcccaagacctcttttcccactttttccctcttcttgactcaccctgccctcaatatcccccggcgcagccagtgaaagggagtccctggctcctggctcgcctgcacgtcccagggcggggagggacttccgccctcacgtcccgctcttcgccccaggctggatggaatgaaaggcacactgtctctctccctaggcagcacagcccacaggtttccaggagtgcctttgtgggaggcctctgggcccccaccagccatcctgtcctccgcctggggccccagcccggagagagccgctggtgcacacagggccgggattgtctgccctaattatcaggtccaggctacagggctgcaggacatcgtgaccttccgtgcagaaacctccccctccccctcaagccgcctcccgagcctccttcctctccaggcccccagtgcccagtgcccagtgcccagcccaggcctcggtcccagagatgccaggagccaggagatggggagggggaagtgggggctgggaaggaaccacgggcccccgcccgaggcccatgggcccctcctaggcctttgcctgagcagtccggtgtcactaccgcagagcctcgaggagaagttccccaactttcccgcctctcagcctttgaaagaaagaaaggggagggggcaggccgcgtgcagccgcgagcggtgctgggctccggctccaattccccatctcagtcgttcccaaagtcctcctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgtgtgtcggccggggccactaggcgctcactgttctctccctccgcgcagccgagccacatcgctcagacaccatggggaaggtgaaggtcggagtcaacgggtgagttcgcgggtggctggggggccctgggctgcgaccgcccccgaaccgcgtctacgagccttgcgggctccgggtctttgcagtcgtatgggggcagggtagctgttccccgcaaggagagctcaaggtcagcgctcggacctggcggagccccgcacccaggctgtggcgccctgtgcagctccgcccttgcggcgccatctgcccggagcctccttcccctagtccccagaaacaggaggtccctactcccgcccgagatcccgacccggacccctaggtgggggacgctttctttcctttcgcgctctgcggggtcacgtgtcgcagaggagcccctcccccacggcctccggcaccgcaggccccgggatgctagtgcgcagcgggtgcatccctgtccggatgctgcgcctgcggtagagcggccgccatgttgcaaccgggaaggaaatgaatgggcagccgttaggaaagcctgccggtgactaaccctgcgctcctgcctcgatgggtggagtcgcgtgtggcggggaagtcaggtggagcgaggctagctggcccgatttctcctccgggtgatgcttttcctagattattctctggtaaatcaaagaagtgggtttatggaggtcctcttgtgtcccctccccgcagaggtgtggtggctgtggcatggtgccaagccgggagaagctgagtcatgggtagttggaaaaggacatttccaccgcaaaatggcccctctggtggtggccccttcctgcagcgccggctcacctcacggccccgcccttcccctgccagcctagcgttgacccgaccccaaaggccaggctgtaaatgtcaccgggaggattgggtgtctgggcgcctcggggaacctgcccttctccccattccgtcttccggaaaccagatctcccaccgcaccctggtctgaggttaa

SEQ ID NO: 46 - DNA, 1975 bp, Homo sapiens
ttaaaacaacagaaacctattgtctcacacttccgggggccagaagtttgaaacccaggtgtgttaggatcctgctccctctgaaggctccagggaagagtgtcctctgctccctccgaaggctccagggaagggtctgtcctcttaggcttctggtggcttgcaggtgcagccctccaatcctcctccccaagcggccttctgcctataaggacacgagtcatactggatgaggggcccactaattgatggcttctgtaaagtccccatctccaaataaggtcacattgtgaggtactgggagttaggactccaacatagcttctctggtggacacaattcaactcctaataacgtccacacaaccccaagcagggcctggcaccctgtgtgctctctggagagcggctgagtcaggctctggcagtgtctaggccatcggtgactgcagcccctggacggcatcgcccaccacaggccctggaggctgcccccacggccccctgacagggtctctgctggtctgggggtccctgactaggggagcggcaccaggaggggagagactcgcgctccgggctcagcgtagccgccccgagcaggaccgggattctcactaagcgggcgccgtcctacgacccccgcgcgctttcaggaccactcgggcacgtggcaggtcgcttgcacgcccgcggactatccctgtgacaggaaaaggtacgggccatttggcaaactaaggcacagagcctcaggcggaagctgggaaggcgccgcccggcttgtaccggccgaagggccatccgggtcaggcgcacagggcagcggcgctgccggaggaccagggccggcgtgccggcgtccagcgaggatgcgcagactgcctcaggcccggcgccgccgcacagggcatgcgccgacccggtcgggcgggaacaccccgcccctcccgggctccgccccagctccgcccccgcgcgccccggccccgcccccgcgcgctctcttgcttttctcaggtcctcggctccgccccgctctagaccccgccccacgccgccatccccgtgcccctcggccccgcccccgcgccccggatatgctgggacagcccgcgcccctagaacgctttgcgtcccgacgcccgcaggtcctcgcggtgcgcaccgtttgcgacttggtgagtgtctgggtcgcctcgctcccggaagagtgcggagctctccctcgggacggtggcagcctcgagtggtcctgcaggcgccctcacttcgccgtcgggtgtggggccgccctgacccccacccatcccgggcgagctccaggtgcgccccaagtgcctcccaggtgttgcccagcctttccccgggcctggggttcctggactaggctgcgctgcagtgactgtggactggcgtgtggcgggggtcgtggcagcccctgccttacctctaggtgccagccccaggcccgggccccgggttcttcctacccttccatgctgccagctttccctccgccagctgctccaggaagcttccagaagcccctgcgcgggccttggcttgcagcaaccctttagcatacttaggcagagtcccatatttccttcctgctggaggccaagttctaggggccttctggttactatggctggtgtttgtgtacatcataccctaactgtattcatcaacacttagagtaagcaaggctcgctggagagccacacacactgggcaccgtaatgtcggttataacaccgcagaggagttctgaactatgtatttcgcactcctgggttcatcatctcctgaaatctcagggtggtgtttgctctcagttgcttcagctgagtagctggctttctgtcctggaaagcagactttgtacatgtgtgtgcaacctatgcctgctgagatcatcatcagacagggaagcggcttggtccagagagctgttctcagtagaatgttaa

SEQ ID NO: 47 - DNA, 1935 bp, Homo sapiens
ttaatacctgggtgatgggatgatctgtacagcaaaccatcatggcgcacacacctatgtaacaaacctgcacatcctctacatgtaccccagaacttcaaataaaagttggacggccaggcgtggtggctcacgcctgtaatcccagcactttgggaagccgaggcgtgcagatcacctaaggtcaggagttcgagaccagcccggccaacatggtgaaaccccgtctctactaaaaatacaaaaatcagccagatgtggcacgcacctataattccacctactcgggaggctgaagcagaattgcttgaacccgagaggcggaggttgcagtgagccgccgagatcgcgccactgcactccagcctgggccacagcgtgagactacgtcataaaataaaataaaataacacaaaataaaataaaataaaataaaataaaataaaataaaataaaataaaataaaataaaaaaataaaataaaataaaataaaataaagcaatttcctttcctctaagcggcctccacccctctcccctgccctgtgaagcgggtgtgcaagctccgggatcgcagcggtcttagggaatttccccccgcgatgtcccggcgcgccagttcgctgcgcacacttcgctgcggtcctcttcctgctgtctgtttactccctaggccccgctggggacctgggaaagagggaaaggcttccccggccagctgcgcggcgactccggggactccagggcgcccctctgcggccgacgcccggggtgcagcggccgccggggctggggccggcgggagtccgcgggaccctccagaagagcggccggcgccgtgactcagcactggggcggagcggggcgggaccacccttataaggctcggaggccgcgaggccttcgctggagtttcgccgccgcagtcttcgccaccagtgagtacgcgcggcccgcgtccccggggatggggctcagagctcccagcatggggccaacccgcagcatcaggcccgggctcccggcagggctcctcgcccacctcgagacccgggacgggggcctaggggacccaggacgtccccagtgccgttagcggctttcagggggcccggagcgcctcggggagggatgggaccccgggggcggggagggggggcagactgcgctcaccgcgccttggcatcctcccccgggctccagcaaacttttctttgttcgctgcagtgccgccctacaccgtggtctatttcccagttcgaggtaggagcatgtgtctggcagggaagggaggcaggggctggggctgcagcccacagcccctcgcccacccggagagatccgaacccccttatccctccgtcgtgtggcttttaccccgggcctccttcctgttccccgcctctcccgccatgcctgctccccgccccagtgttgtgtgaaatcttcggaggaacctgtttccctgttccctccctgcactcctgacccctccccgggttgctgcgaggcggagtcggcccggtccccacatctcgtacttctccctccccgcaggccgctgcgcggccctgcgcatgctgctggcagatcagggccagagctggaaggaggaggtggtgaccgtggagacgtggcaggagggctcactcaaagcctcctgcgtaagtgaccatgcccgggcaaggggagggggtgctgggccttagggggctgtgactaggatcgggggacgcccaagctcagtgcccctccctgagccatgcctcccccaacagctatacgggcagctccccaagttccaggacggagacctcaccctgtaccagtccaataccatcctgcgtcacctgggccgcacccttggtgagtcttgaacctccaagtccagggcaggcatgggcaagcctctgcccccggagcccttttgtttaa

SEQ ID NO: 48 - DNA, 937 bp, Homo sapiens
ttaaccttactcgccccagtctgtcccgacgtgacttcctcgaccctctaaagacgtacagaccagacacggcggcggcggcgggagaggggattccctgcgcccccggacctcagggccgctcagattcctggagaggaagccaagtgtccttctgccctcccccggtatcccatccaaggcgatcagtccagaactggctctcggaagcgctcgggcaaagactgcgaagaagaaaagacatctggcggaaacctgtgcgcctggggcggtggaactcggggaggagagggagggatcagacaggagagtggggactaccccctctgctcccaaattggggcagcttcctgggtttccgattttctcatttccgtgggtaaaaaaccctgcccccaccgggcttacgcaatttttttaaggggagaggagggaaaaatttgtggggggtacgaaaaggcggaaagaaacagtcatttcgtcacatgggcttggttttcagtcttataaaaaggaaggttctctcggttagcgaccaattgtcatacgacttgcagtgagcgtcaggagcacgtccaggaactcctcagcagcgcctccttcagctccacagccagacgccctcagacagcaaagcctacccccgcgccgcgccctgcccgccgctgcgatgctcgcccgcgccctgctgctgtgcgcggtcctggcgctcagccatacaggtgagtacctggcgccgcgcaccggggactccggttccacgcacccgggcagagtttccgctctgacctcctgggtctatcccagtactccgacttctctccgaatagagaagctacgtgacttgggaaagagcttggaccgctagagttcgaaagaactccgtggatattccagctttcccacaagcactgatcattatgagccagttacttaa

SEQ ID NO: 49 - DNA, 704 bp, Homo sapiens
ttaatagcacctcctccgagcactcgctcacggcgtccccttgcctggaaagataccgcggtccctccagaggatttgagggacagggtcggagggggctcttccgccagcaccggaggaagaaagaggaggggctggctggtcaccagagggtggggcggaccgcgtgcgctcggcggctgcggagagggggagagcaggcagcgggcggcggggagcagcatggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtgggtagagggtctgcagcgggagcaggggatggcgggcgactctggaggacgaagtttgcaggggaattggaatcaggtagcgcttcgattctccggaaaaaggggaggcttcctggggagttttcagaaggggtttgtaatcacagacctcctcctggcgacgccctgggggcttgggaagccaaggaagaggaatgaggagccacgcgcgtacagatctctcgaatgctgagaagatctgaaggggggaacatatttgtattagatggaagtatgctctttatcagatacaaaatttacgaacgtttgggataaaaagggagtcttaa

SEQ ID NO: 50 - DNA, 1238 bp, Homo sapiens
ttaaaggctgcggactgtgctactgccccttctgatgccccctcctctacacagcaatcattcagcgtcccttagtcactccggacagcgacaggccccgcggccgccatgcccaccgcctccatgccatgcccaccgccgccatgcctaccgccgccaaagtccaccaccgccatgcctacccgctgccaatgcccaccgccgccaatacccactgtcgccgccttccccctacctcccagccacttcctacggactctccccgcgccgcgaccaccaacacaacccccaccactgtcacaccgactcatccccctggtccactgccatagcctcctcgcctcggtcactgcgacgaattccccccccagtcgccccacgtaccctgctccaccacgcagtggtcactattatacacctacctgcgctcaacaccccctaaataccgatcacttcacgtaccttcgccccgccacaatcactccaatatacctacctccgcctaaaatccctatgcactggtccccccacgtaccctcgccacacggaactgcaatcaccctgatgtacccacctccacccatgtcccttgcccactgcggttaccccgcatgctcccagtcaccaccgcccttcccaccgcagacacccgcaataggacctgtcgcgacaccacagttgggggcggatgggggacgcgccccaatgcgagcggacaggataccatcggggcagaacggcacaacagcaagcctctgaacattccggatctggttctccagaacaaaggactttagggcccaaattccgtttattcagtactccaagtcctaaaaacttggaatatctgatgaataaaagtggccgctccccaggctgtctcttgagagaagccaccggcacagctgaccttgcccgctccatcgcgtcactgaccgctcctcagacagatgcgtcaggcatctccggcggccgctccactctgcgccagactcgctgcagcagcggcaggcttcgcacacatccccgcctgagcatgcgcgccagcctgcctctgcggccgcgcaggcgtgcttgtttgccgcagtgcaggggtcccagctccctccctcaccggaatgacctggggggagggggctactggacccctagggccccacagcactgttgcaatgagagggggcctctagaaaccataagcaacctgggatcaatggacatgtctacctgttttttaa

SEQ ID NO: 51 - DNA, 20 bp, Artificial Sequence
  Synthetic primer
tgcatagggattttaggcgg

SEQ ID NO: 52 - DNA, 21 bp, Artificial Sequence
  Synthetic primer
ccgatcacttcacgtaccttc

SEQ ID NO: 53 - DNA, 22 bp, Artificial Sequence
  Synthetic Primer
acctccgcctaaaatccctatg

SEQ ID NO: 54 - DNA, 20 bp, Artificial Sequence
  Synthetic Primer
cttgctgttgtgccgttctg

SEQ ID NO: 55 - DNA, 20 bp, Artificial Sequence
  Synthetic primer
aggcactccctcacggggtc

SEQ ID NO: 56 - DNA, 20 bp, Artificial Sequence
  Synthetic primer
gagggctgcgggcgaactag

SEQ ID NO: 57 - DNA, 20 bp, Artificial Sequence
  Synthetic primer
gcccgcagggaccgcttcaa

SEQ ID NO: 58 - DNA, 21 bp, Artificial Sequence
  Synthetic primer
ccttcaccaccgccacgttgt

SEQ ID NO: 59 - DNA, 22 bp, Artificial Sequence
  Synthetic primer
tgtagcagccttagtcgccgcc

SEQ ID NO: 60 - DNA, 20 bp, Artificial Sequence
  Synthetic primer
gcgcactcaccaaccccgta

SEQ ID NO: 61 - DNA, 24 bp, Artificial Sequence
  Synthetic primer
attgattcagcaacttggcctgcg

SEQ ID NO: 62 - DNA, 24 bp, Artificial Sequence
  Synthetic primer
tttgtgcaacgaaccctgactcct

SEQ ID NO: 63 - DNA, 21 bp, Artificial Sequence
  Synthetic primer
cggcgcttctcctcctgatgc

SEQ ID NO: 64 - DNA, 20 bp, Artificial Sequence
  Synthetic primer
cagccctcggactcacgcag

SEQ ID NO: 65 - DNA, 24 bp, Artificial Sequence
  Synthetic primer
tttgattatcgagggcgctggcgt

SEQ ID NO: 66 - DNA, 22 bp, Artificial Sequence
  Synthetic primer
acgtgtgtgggtctcgtaaggt

SEQ ID NO: 67 - DNA, 20 bp, Artificial Sequence
  Synthetic primer
ggatcccagcgccgaaccag

SEQ ID NO: 68 - DNA, 20 bp, Artificial Sequence
  Synthetic primer
cttccgcgtccactgagccg

SEQ ID NO: 69 - DNA, 20 bp, Artificial Sequence
  Synthetic primer
actgaggctgggaggtcggc

SEQ ID NO: 70 - DNA, 20 bp, Artificial Sequence
  Synthetic primer
cgggcactagcggagccaag

SEQ ID NO: 71 - DNA, 20 bp, Artificial Sequence
  Synthetic primer
ggggcatccattgcccggag

SEQ ID NO: 72 - DNA, 20 bp, Artificial Sequence
  Synthetic primer
ggaagcccgaggagcctgga

SEQ ID NO: 73 - DNA, 20 bp, Artificial Sequence
  Synthetic primer
tctcgggactcacccgagcg

SEQ ID NO: 74 - DNA, 1242 bp, Homo sapiens
ttaaaggctgcggactgtgctactgccccttctgatgccccctcctctacacagcaatcattcagcgtcccttagtcactccggacagcgacaggccccgcggccgccatgcccaccgcctccatgccatgcccaccgccgccatgcctaccgccgccaaagtccaccaccgccatgcctacccgctgccaatgcccaccgccgccaatacccactgtcgccgccttccccctacctcccagccacttcctacggactctccccgcgccgcgaccaccaacacaacccccaccactgtcacaccgactcatccccctggtccactgccatgcctcctcgcctcggtcactgcgacgaattccccccccagtcgccccacgtaccctgctccaccacgcagtggtcactattatacacctacctgcgctcaacaccccctaaataccgatcacttcacgtaccttcgccccgccacaatcactccaatatacctacctccgcctaaaatccctatgcactggtccccccacgtaccctcgccacacggaactgcaatcaccctgatgtacccacctccaccatgtcccttgcccactgcggttaccccgcatcccgcatgctcccagtcaccaccgcccttcccaccgcagacacccgcaataggacctgtcgcgacaccacagttgggggcggatgggggacgcgccccaatgcgagcggacaggataccatcggggcagaacggcacaacagcaagcctctgaacattccggatctggttctccagaacaaaggactttagggcccaaattccgtttattcagtactccaagtcctaaaaacttggaatatctgatgaataaaagtggccgctccccaggctgtctcttgagagaagccaccggcacagctgaccttgcccgctccatcgcgtcactgaccgctcctcagacagatgcgtcaggcatctccggcggccgctccactctgcgccagactcgctgcagcagcggcaggcttcgcacacatccccgcctgagcatgcgcgccagcctgcctctgcggccgcgcaggcgtgcttgtttgccgcagtgcaggggtcccagctccctccctcacggaatgacctggggggagggggctactggacccctagggccccacagcactgttgcaatgagagggggcctctagaaaccataagcaacctgggatcaatggacatgtctacctgttttttaa

(19) United States
(12) Patent Application Publication
(10) Pub. No.: US 2013/0157266 A1
(43) Pub. Date: Jun. 20, 2013

(54) ABSCRIPTION BASED MOLECULAR DETECTION OF DNA METHYLATION

(71) Applicants: Michelle M. Hanna, Carlsbad, CA (US); David McCarthy, Carlsbad, CA (US)

(72) Inventors: Michelle M. Hanna, Carlsbad, CA (US); David McCarthy, Carlsbad, CA (US)

(73) Assignee: RIBOMED BIOTECHNOLOGIES, INC., Carlsbad, CA (US)

(21) Appl. No.: 13/609,222

(22) Filed: Sep. 10, 2012

Related U.S. Application Data
(63) Continuation-in-part of application No. 12/724,416, filed on Mar. 15, 2010, now Pat. No. 8,263,339.
(60) Provisional application No. 61/160,335, filed on Mar. 15, 2009.

(51) Int. Cl. C12Q 1/68

(52) U.S. Cl. CPC C12Q 1/6886; C12Q 1/6883
USPC 435/6.11

(57) ABSTRACT

The present invention provides methods for detecting biomarkers based on Abscription®, abortive transcription technology. Particularly, the present invention provides bisulfate free methods for detecting methylation of CpG islands from small samples containing DNA, including formalin fixed, paraffin embedded samples. The methods are suitable for multiplexing and can be used to analyze multiple CpG islands from a single sample in a short time.

### RELATED APPLICATION

This application is a continuation in part of U.S. patent application Ser. No. 12/724,416, filed Mar. 15, 2010, now U.S. Pat. No. 8,263,339, issued Sep. 11, 2012, which in turn claims the benefit of priority under 35 USC §119 of U.S. Provisional Application Ser. No. 61/160,335 filed Mar. 15, 2009, the entire disclosures of which are incorporated herein by reference.

### STATEMENT OF GOVERNMENT INTEREST

This invention was made with government support under from the National Institutes of Health through National Cancer Institute Small Business Innovative Grant number 1R43CA132851-1 and NCl Contract number HHSN261200900047C. The Government may have certain rights in the invention.

### BACKGROUND OF THE INVENTION

Cancer is actively avoided through the expression of numerous tumor-suppressor genes that regulate the cell division cycle and mediate interactions among cells. Studies of benign and malignant tumors have shown that cancer develops through a multi-step process where randomly accumulated changes either enhance the expression of proto-oncogenes or reduce the expression or function of tumor-suppressor and DNA repair genes. Somatic mutations account for some of these changes in tumor-suppressor and DNA repair genes. However, it has recently become apparent that epigenetic changes, such as DNA hypermethylation and hypomethylation, also play a large role in the development of cancer through inactivation or enhancement of tumor suppressors or proto-oncogenes. Hypermethylation of CpG promoter islands occurs at an early stage of cancer development and is found in virtually all tumors, making it potentially very useful as a diagnostic marker, allowing cancer to be noninvasively detected in the early stages when treatment is most effective. For example, hypermethylation of the promoter region of genes such as DAP kinase, p16, and MGMT has reportedly been detected in the sputum of smokers up to 3 years prior to the diagnosis of squamous cell lung carcinoma (Belinsky et al. (2006). Cancer Res. 66:3338-44, Palmisano et al. 2000. Cancer Res. 60:5954-8). Similarly, hypermethylation of a small panel of genes may be a valuable early detection indicator for non-small cell lung cancer. Hypermethylation of a small panel of genes was detected in the early stages of breast cancer but was not detected in normal or benign breast tissue Krassenstein et al. (2004). Clin Cancer Res. 10:28-32.

Methylation of cytosines in CpG islands is an early event in most cancers that leads to reduced expression of many genes, including tumor suppressor genes. Surveys of CpG island methylation in tumor cell DNA suggest that this epigenetic change is common enough to rival the impact of mutation in tumor progression. Early detection of abnormal methylation can lead to regular screening and early diagnosis when treatment is most effective. Early epigenetic changes are often detectable in blood serum or other bodily fluids (urine, sputum, saliva), not just in the tumor tissue itself, which means that epigenetic diagnostic testing can be done noninvasively using these fluids. Diagnostic tests based on CpG island methylation may also be utilized for drug development, identification of patient populations that will respond to these drugs and post treatment monitoring.

Recent improvements in the sensitivity of methylation detection and elucidation of methylation signatures for specific cancers have made it feasible to assay for tumors by sampling DNA from bodily fluids. Tumor cells release DNA into blood from a relatively early stage of the disease. From 3% to over 90% of the DNA in the blood of cancer patients has been found to be of tumor origin (Kim et al. (2004) J. Clin. Oncol. 22:2363-70. The development of PCR-based methylation assays has made it possible to detect the presence of tumors noninvasively from blood, sputum, and urine. In one study methylation detection was more sensitive than urine cytology in detecting aberrant premalignant cells. Clinical sensitivities (the proportion of confirmed cases detected) are typically low in surveys of blood samples using a single methylation marker. However, clinical specificity (the proportion of normal controls that test negative) from blood samples approaches 100%. The clinical sensitivities should increase with methylation tests that assess more than a single CpG island.

The appearance of abnormally methylated DNA in bodily fluids by itself does not help to pinpoint which organ is affected by a tumor. Surprisingly little information is needed to establish the tissue origin of a tumor. Numerous methylation screening studies have established methylation signatures; collections of methylated CpG islands that are strongly associated with cancers from particular organs. In one survey, aberrant methylation of 3 to 4 candidate CpG islands was sufficient to identify from 70-90% of 15 cancer types (Esteller et al. (2001) Cancer Res. 61:3225-9). Methylation profiles developed for primary tumors could be applied to tumor cell lines to accurately identify the tissue origin of their parent tumors (Graziano et al. (2004) Clin. Cancer Res. 10:2784). This result suggests that methylation signatures of different tumor types are not greatly affected by the selective pressures associated with growth in culture. The availability of methylation profiles should greatly enhance the ability to detect cancer in inaccessible organs through a simple blood test.

Detection of methylation in clinical samples would enable early detection of cancer. The development of simple and sensitive multiplex detection assays will allow small clinical samples to be profiled for the status of multiple CpG islands. This kind of information will be valuable in diagnosis and treatment.

### Methods for Detecting DNA Methylation

A number of methods have been used to detect methylated-CpG (mCpG) in target DNA. The three primary methods in current use are detailed below.

Bisulfite Methods.

The most commonly used methylation detection methods are based on bisulfite modification of DNA, resulting in deamination of cytosine residues to uracil while leaving the methylated cytosines unchanged. Upon PCR amplification, the methylated cytosine is copied to cytosine and uracil is copied to thymine. As a result, the retention of cytosine at a specific position indicates methylation. The modified DNA is then analyzed, e.g. by sequence analysis, methylation-specific PCR (MSP) (Herman et al. (1996) Proc. Natl. Acad. Sci. USA 93:9821-26), or hybridization (e.g. to a microarray or blot). In MSP, a pair of methylation-specific oligonucleotide primers is added to the bisulfite-treated DNA and PCR is performed in order to amplify the target DNA. Fluorescence-based quantitative real-time PCR can also be performed on bisulfite-modified DNA (Eads et al. (2000) Nucl. Acids Res. 28:E32; Zeschnigk et al. (2004) Nucl. Acids Res. 32:e125).

Calibrated, fluorescence-based variants of MSP exploit real-time PCR to provide quantification of the amount of methylated DNA in a sample. An important underlying assumption of these PCR based methods is that the few CpG sites that are recognized by the primers/probes reflect the overall status of the target CpG island. While this is usually true for heavily methylated or completely unmethylated islands, partially methylated targets are probably not readily scored in methylated- or unmethylated-specific reactions.

An advantage of bisulfite modification is that it differentially marks methylated versus unmethylated sites allowing sequencing methods to detect methylation patterns. Sequencing of cloned bisulfite-treated DNA is the most commonly used method for methylation detection. It provides information on the success of the bisulfite treatment in addition to sampling a greater number of CpG sites than the MSP based methods. Due to its complexity and expense, however, bisulfite sequencing is better suited for marker discovery than clinical diagnostics. Bisulfite treatment destroys a large percentage of the input DNA, resulting in limited sensitivity and a requirement for large amounts of DNA. Quality control assessments of bisulfite treated DNA are necessary before performing a detection assay to avoid misleading results. There is a potential of false-positive results for MSP-based assays due to incomplete cytosine deamination during bisulfite treatment. Amplification of bisulfite treated DNA is affected by PCR bias favoring unmethylated DNA. While this problem can usually be corrected by optimizing primer annealing conditions, it may complicate primer design and testing. Template biases can be eliminated with the use of digital bisulfite-PCR. Dilution of the DNA sample to an average of less than one copy per reaction eliminates competition among templates. Individual molecules can be sequenced without biases introduced by cloning.

Commercial kits, reagents and systems employing bisulfite treatment for analyzing mCpG are available. Epigenetics (Berlin) offers two variants of the MethyLight assay, adaptations of quantitative real-time PCR, called Quantitative MethyLight (QM) and Heavy Methyl (HM). QM utilizes Taqman® probes to generate a fluorescent signal. During the course of amplification, the fluor is cleaved from the Taqman® probe resulting in fluorescence that can be detected in real-time (Wojdacz & Dobrovic (2007) Nucl. Acids Res. 35:e41). HM is an adaptation of QM in which blocker oligonucleotides are added to the reaction. These blocker oligonucleotides prevent amplification of unmethylated DNA, resulting in increased assay sensitivity (Cottrell et al. (2004) Nucl. Acids Res. 32:e10). Pyrosequencing® is also utilized for methylation quantification from bisulfite-modified DNA, as exemplified by the Pyro Q-CpG™ system from Biotage (Uppsala, Sweden; Tost et al. (2003) Biotechniques 35:152-56).

Although bisulfite modification is a widely used, the extensive DNA degradation it causes can introduce sampling errors when few molecules are long enough to be amplified (Ehrich et al. (2007) Nucl. Acids Res. 35:e29). Furthermore, the assays are time-consuming, require a harsh base denaturation step, and have a high-probability of false-positive results due to incomplete cytosine deamination during bisulfite treatment.

Methylation-Sensitive Restriction Enzyme Digestion Methods.

A second type of method for detecting mCpG in DNA relies on differential cleavage by restriction endonucleases. DNA is treated with either a MSRE (methylation-sensitive restriction endonuclease) or a MDRE (methylation dependent restriction endonuclease), amplified and then analyzed by microarray or gel electrophoresis. MSREs such as HpaII and AciI cut a DNA sequence only if it is unmethylated. MDREs are restriction endonuclease that require methylation of a DNA sequence for cleavage. By treating a sample of DNA with either of these enzymes and subsequent comparison to a control sample, the methylation state of the DNA sample can be determined. If digestion of a specific DNA sample occurs after treatment with a MDRE, then the DNA can be assumed to be methylated. Conversely, if the DNA is uncut when treated with a MSRE, then the sample can be assumed to be methylated. By comparing the amount of cut versus uncut DNA, the level of methylation can be estimated. A common read-out for this type of methylation analysis is the subsequent amplification and fluorescent labeling of the digested DNA. The fragments can then be hybridized to a library microarray and analyzed or simply resolved by electrophoresis.

Commercially available restriction endonuclease-based systems include Orion's MethylScope, which utilizes a microarray read-out (Lippman et al. (2004) Nature 430:471-76), and MethyScreen, which employs quantitative real-time PCR (Ordway et al. (2006) Carcinogenesis 27:2409-23).

An advantage of MSRE/MDRE digestion is that no pre-treatment of the DNA is necessary, although it is often performed in conjunction with bisulfite treatment of DNA in a procedure called COBRA (Xiong & Laird (1997) Nucl. Acids Res. 25:2532-34). Some disadvantages with this procedure are that it is lengthy and is dependent on the presence of MSRE/MDRE recognition sequences within a target DNA. Furthermore, this approach is relatively inefficient, which can reduce the reliability of the results. The only CpG sites that are assessed are those within a small number of restriction enzyme recognition sites and status of those sites may not reflect the status of the entire CpG island in which the site reside. Incomplete digestion leads to frequent false positives, especially when cleavage reactions are subjected to a subsequent amplification step. Restriction endonuclease cleavage assays have poor sensitivity compared to bisulfite methods, such as MSP, allowing detection of not less than 10% methylated DNA in a sample (Singer-Sam et al. Nucleic Acids Res, 1990. 18:687; Yegnasubramanian et al. Nucleic Acids Res. (2006) 34:e19).

Chromatin Immunoprecitipation Methods.

A third method that is commonly employed for detecting mCpG is chromatin immunoprecipitation (ChIP). Typically, cells are fixed, and then methylated DNA is immunoprecipitated by the use of antibodies specific for methyl binding proteins. The resulting DNA is amplified, labeled and analyzed by hybridization in a microarray assay. The advantages of this method are that the assay can be performed from live cells with little or no DNA purification required. The assay also has increased sensitivity, as unwanted and contaminant DNA are removed prior to analysis. However, the ChIP procedure is very time-consuming, involves several steps and requires expensive reagents. Some assays may take as long as five days to complete.

Methods Using Methyl Binding Proteins.

An alternative and more sensitive approach to separating methylated from unmethylated DNAs involves the use of methyl-CpG binding domain (MBD) proteins or antibodies against 5-methyl-C. MBD proteins have high affinity for methylated CpG sites and very low affinity for unmethylated DNA (Fraga et al. Nucleic Acids Res. (2003) 31:1765-74). Samples are incubated with immobilized MBD protein in a variety of formats (magnetic beads, columns, the walls of PCR tubes). Methylated DNA capture is usually followed by amplification of the captured DNA. MBD-based DNA detection has the major advantage that all of the methylated sites can contribute to binding, thereby allowing an entire island to be sampled for methyl-CpGs. This characteristic makes the binding assay less vulnerable to false negatives that affect MSP and restriction endonuclease-based assays when unmethylated sites in a partially methylated island correspond to priming/probe sites (Yegnasubramanian et al., Nucleic Acids Res. (2006) 34:e19). This situation is likely to be common in clinical samples containing early stage tumor cells that contain partially methylated CpG islands. MBD based binding assays are very sensitive, allowing detection of as little as 160 pg of methylated DNA (equivalent to ˜25 cells) or 1 methylated molecule in 500 unmethylated molecules (Gebhard et al. Nucleic Acids Res. (2006) 34:e8256). This is close to the sensitivity of MSP (1 methylated molecule/1,000 unmethylated molecules). The COMPARE MBD assay can be as sensitive as real-time MSP (1 methylated molecule/10,000 unmethylated molecules) by including digestion with HpaII (an MSRE) before the binding step. Cleavage of unmethylated DNAs at a location between PCR priming sites gives high sensitivity with DNA mixtures that contain artificially methylated DNAs that are fully methylated (Yegnasubramanian et al. Nucleic Acids Res. (2006) 34:e19). However this strategy could suffer the disadvantage associated with the use of restriction endonucleases in that some partially methylated islands will be scored as unmethylated in clinical samples (Yegnasubramanian, et al. supra).

Imprinting Disorder Diagnosis by Analysis of DNA Methylation.

Genetic alterations in DNA methylation that affect imprinting play important roles in Prader-Willi syndrome (PWS) and Angelman syndrome (AS) and other diseases and syndromes. The imprinted genes in chromosome 15 region 15q11.2-q13 that are associated with PWS and AS, including the SNRPN (small nuclear ribonucleoprotein peptide N) promoter region, are normally methylated and unexpressed in the maternal chromosome and unmethylated and expressed in the paternal chromosome. Loss of the unmethylated and expressed paternal copy by deletion, maternal uniparental disomy (UDP) or by imprinting errors, leaving the methylated and unexpressed maternal copy as the only version of the gene, is associated with PWS due to loss of paternal expression. Conversely, AS is associated with the loss of the maternal copy of 15q11.2-q13 which can occur through deletion, mutation in the maternally expressed gene Ubiquitin-protein ligase E3A (UBE3A) or paternal UDP. Methylation analysis of the SNRPN promoter is used to confirm diagnosis of PWS although methylation studies alone do not define the genetic basis for the diagnosis. A positive result of a methylation analysis leads to follow-up studies to define the genetic cause.

The most commonly used diagnostic methylation tests for PWS and AS are MSP and Southern blot assays using methylation sensitive restriction enzymes. Methylation-specific multiplex ligation dependent amplification (MS-MLA) has also been used to identify the methylation status of the PWS region and to detect copy number changes in the region. The Southern blot assay and one version of MS-MLA depend on the use of methylation sensitive restriction enzymes which probe the methylation status of one or more CpG sites. Results can be affected by incomplete digestion of genomic DNA or rare SNPs affecting restriction sites. MSP and alternative versions of MS-MLA use bisulfite treated DNA.

Given the importance of CpG methylation in cancer development and progression, and in disease-related imprinting, a rapid, reliable, and sensitive test for methylated CpG DNA would provide an important and useful clinical tool.

### SUMMARY OF THE INVENTION

The present invention provides methods for detecting target polynucleotides in a sample. The methods generally involve contacting a sample containing a target polynucleotide with a primer pair that specifically hybridizes to and amplifies a target sequence of the polynucleotide. For this step, the primer pair includes a first primer with a 3′ sequence complementary to a first sequence flanking the target polynucleotide sequence, and a 5′ capture tag. The second primer of the pair has a 3′ sequence complementary to a second sequence flanking the polynucleotide target sequence on the opposite strand, and a 5′ sequence that provides a means for directing Abscription. Following amplification (e.g. PCR) using this primer pair, the amplified target sequence is contacted with an immobilized molecule that binds the 5′ capture tag, to capture the amplified target sequence. At least one Abscript is then transcribed from the means for directing Abscription and the Abscript detected as an indication of the presence of the target polynucleotide.

Capture will typically be via an affinity reagent or binding pair bound or capable of being bound to a solid support. For example, the 5′ capture tag can be biotin, which can be readily incorporated into oligonucleotide primers, and the molecule that binds to the 5′ capture tag can be streptavidin immobilized on a solid support. A wide variety of solid supports are suitable for use in the methods of the present invention, such as beads, tubes, and microtiter plates. Conveniently, steptavidin and other binding pair molecules can be bound to magnetic beads which permit rapid separation of the solid phase from unbound reagents in solution. In certain embodiments of the invention, unbound reagents, primers, and polynucleotides can be washed from immobilized and captured polynucleotides prior to the subsequent steps in the procedure, which may increase the efficiency of the method. However, this is not necessary as the entire method can be performed in a single pot or tube without separation steps.

PCR is typically used for the amplification step, using for example, a thermostable DNA polymerase or a thermostable RNA polymerase. However, a variety of target amplification methods known in the art may be suitable for use in the methods of the present invention

A variety of methods are available for detecting Abscripts as described herein, including, but not limited to mass spectrometry, capillary electrophoresis or thin layer chromatography. In certain aspects, a detectably labeled nucleotide or other label can be incorporated into Abscript signals generated by the methods of the invention to increase the sensitivity or expand the detection techniques that may be used. For example, the detectably labeled nucleotide can be a fluorescent nucleotide.

Abscripts generated by the present invention will generally be short, e.g. 3-20 nucleotides in length. Abscripts as small as 3 nucleotides in length are typically used in the methods described herein.

The second primer of the pair used during amplification has a 3′ sequence complementary to a second sequence flanking the target sequence of the polynucleotide on the opposite strand, and a 5′ sequence that provides a means for directing Abscription.

In certain embodiments, the means is provided by an α-TAP (Target Attachment Probe) sequence that is used to tag or identify the target. The α-TAP is designed to be complementary to a TAP sequence and permits the attachment of an APC (Abortive Promoter Cassette). To maintain the α-TAP as a single-strand that is thereby available for hybridization to the a TAP sequence, a non-natural nucleotide can be included between the 5′ α-TAP sequence and the 3′ sequence complementary to the sequence flanking the target in the primer. Non-natural nucleotides, such as etheno-deoxyadenosine, are not recognized by polymerases during amplification. Thus, sequences downstream from the non-natural nucleotide in a primer are not replicated and those sequences remain single-stranded.

Once an APC is bound to the amplified target through the TAP-α-TAP hybrid that is formed, Abscripts are transcribed from the APC as an indication or signal for detecting the presence of the target. The APC that is bound is either a double-strand region or is made double stranded by hybridization of a probe.

In certain embodiments of the invention, a fully duplex APC can be generated during the amplification reaction from a primer sequence that includes one strand of the APC. Conveniently, Abscription can be performed during the amplification by including a thermostable RNA polymerase and nucleotides in the reaction. Thus, in these embodiments, the second primer for the amplification reaction includes an APC sequence. As duplex APCs are generated (e.g. by PCR), Abscripts are transcribed from the APC and can be detected as they are produced (e.g. in real-time), or analyzed at a later time by Abscript detection methods described herein.

The invention provides rapid, sensitive, and specific methods for detecting a variety of variety of target polynucleotides of interest, including DNA and RNA targets, with an expanded repertoire of detection techniques as compared to PCR. Unlike PCR, the methods are also suitable for detecting polynucleotide targets that are modified, such as methylated DNA targets. According to such methods, methylated genomic DNA fragments are first isolated by cleaving a genomic DNA sample containing a methylated target polynucleotide (such as a CpG island), with a restriction enzyme that does not cleave the target polynucleotide, or generates suitably representative fragments of the target for during cleavage. The cleaved genomic DNA is then contacted with an immobilized methyl binding domain, such as the GST-MBD2 fusion protein described herein. In this way, methylated genomic DNA fragments are immobilized and therefore isolated from the non-methylated DNA fragments in the sample. Optionally, the methylated genomic DNA fragments can be eluted from the immobilized MBD and recovered prior to analysis. For example, where the GST-MBD2 fusion protein is used for immobilizing methylated CpG island targets, the GST portion of the fusion protein can be bound to a glutathione resin (before or after interaction with DNA), and the bound methylated DNA fragments can be eluted with glutathione.

The methods of the invention are also suitable for multiplexing. According to certain embodiments of the invention, the a plurality of different target polynucleotides, are processed simultaneously by including a plurality of first and second primer pairs in the reactions, each primer pair being designed to specifically hybridize to a different target polynucleotide. By designing different, unique APCs for each target that are attached to the target through the primed amplification (either as part of the APC-containing primer or via the TAP-α-TAP hybrid, as described herein), the presence of each of the plurality of target can be identified through the APC signal that is generated. For example, each APC can be designed to be distinguishable on the basis of molecular weight or nucleotide sequence. According to these embodiments of the invention, at least 5, 10, 20, 50, 100 or more targets can be detected in a single assay.

In certain embodiments of the invention, methods are provided for detecting methylation of a target polynucleotide in a sample comprising the steps of cleaving a genomic DNA sample containing at least one methylated target polynucleotide with a restriction enzyme, such as a restriction enzyme that does not cleave the target polynucleotide CpG island; contacting the cleaved genomic DNA with an immobilized MBD (such as GST-MBD2), thereby immobilizing methylated genomic DNA from the sample; optionally, recovering the methylated genomic DNA from the immobilized MBD, thereby isolating methylated genomic DNA fragments; contacting the methylated DNA with a primer pair that specifically hybridizes to and amplifies a target sequence of the at least one polynucleotide; and detecting the amplified polynucleotide. In certain aspects of the invention, the MBD, such as a GST-MBD2 fusion protein, is immobilized on a glutathione-containing solid support.

Optionally, the unmethylated (unbound) DNA can also be collected and the unmethylated target DNA detected in a similar fashion. The primer pair, in certain aspects of the invention will include a first primer comprising a 3′ sequence complementary to a first sequence flanking the target sequence of the polynucleotide, and a second primer comprising: a 3′ sequence complementary to a second sequence flanking the target sequence of the polynucleotide, and a 5′ sequence comprising one strand of an APC. Amplification with the primer pair thereby generates an APC.

Dection of the amplified polynucleotide can include amplifying the target sequence from the first and second primers, wherein the amplification produces an APC; transcribing at least one Abscript from the APC; and detecting the at least one transcribed Abscript.

At various steps in the process, the unbound reagents and polynucleotides can be washed from immobilized reagents, and optionally collected for detection of the target polynucleotide. For example, the unbound polynucleotides comprising unmethylated DNA can be recovered, contacted with a primer pair that specifically hybridizes to and amplifies a target sequence of the at least one polynucleotide (which can be the same primers and target as detected in the methylated DNA), and detecting the amplified unmethylated polynucleotide.

Amplifying can be by any suitable method but will typically consists of performing a polymerase chain reaction, such as a polymerase chain reaction is performed with at least one of a thermostable DNA polymerase and a thermostable RNA polymerase. In certain aspects of the invention, a detectably labeled nucleotide, such as a fluorescent nucleotide, is incorporated into the at least one Abscript during Abscription. Detecting the at least one Abscript, which can be, e.g., 3-20 nucleotides in length, can be by mass spectrometry, capillary electrophoresis or thin layer chromatography.

In certain embodiments, the at least one target polynucleotide is a methylated CpG island and the sample comprises isolated methylated genomic DNA fragments. In yet further aspects, the at least one polynucleotide is differentially methylated in cancer or in an imprinting related disease, disorder or syndrome.

The methods of the invention can be performed using a variety of samples containing DNA, including but not limited to genomic DNA in samples of formalin fixed, paraffin embedded tissue.

### BRIEF DESCRIPTION OF THE DRAWINGS

FIG. 1 illustrates the process of abortive transcription, which is exploited by the Abscription® methods of the invention. Abortive transcription occurs on most promoters when RNA polymerase (RNAP) is trapped at the promoter repeatedly making short abortive transcripts (typically 2 to 12 nt long). During abortive transcription, the RNAP does not translocate or leave the promoter. During normal transcription, RNAP eventually undergoes a conformational change to a stable, processive elongation complex, a process called promoter escape, and then continues transcription until a termination signal is reached. Artificial promoters, or Abortive Promoter Cassettes (APCs) have been developed that trap RNAP in an abortive complex, reiteratively synthesizing thousands of identical short oligonucleotides per minute. Each APC is designed to make a different Abscript of specific length and sequence which can be separated and quantified.

FIG. 2 illustrates detection of protein, RNA and DNA targets using Abscription®. APCs are attached to a Target Attachment Probe (TAP), which specifically binds to the molecular target (FIG. 2A). For detection of DNA and RNA, TAPs include oligonucleotides that specifically hybridize to or proteins that bind specifically to the nucleic acid target (FIG. 2B). For protein detection, APCs can be attached to any molecule that binds to the protein target, such as an antibody or a ligand. FIG. 2C illustrates an embodiment of the invention where the APC is attached to an antibody. The target protein can be “sandwiched” between an APC-antibody complex and a second antibody immobilized on a solid support, for capture and detection of the protein targets similar to strategies used in enzyme linked immunosorbent assays (ELISA).

FIG. 3 illustrates dinucleotide initiation of Abscription® and termination after the addition of one NTP. Abscript length can be limited by inclusion of chain terminating NTPs (3′-O-Me-NTPs at R3) as depicted, or by omitting one or more NTPs from the reaction. R1=Affinity tag, Fluorescent Tag; R2=OH, OMe, H; R3=OH, OMe, H; R4=OH, OMe, H.

FIGS. 4A and 4B illustrate detection of Abscripts by mass spectrometry. An Abscription® reaction that included the initiator GpA and GTP was fractionated by reverse-phase HPLC. The output of the column was introduced into a mass spectrometer. FIG. 4A shows the column profile in terms of total ion count as a function of retention time. FIG. 4B shows the ion spectrum for the trinucleotide Abscript GAG peak (retention time 5.4 min). Singly- and doubly-charged GAG species have m/z values of 956.1 and 477.6, respectively. The sodium adduct of the double charged species has a m/z of 978.2.

FIG. 5 is an illustration of the structure of a GST-MBD Protein used in methylation detection methods of the invention. FIG. 5 A shows the linkage of a MBD domain to the carboxyl end of a GST domain. The amino acid sequence linking the domains (SEQ ID NO:1) contains a thrombin cleavage site indicated by the arrow. FIG. 5B shows the amino acid sequence for the DNA binding domain of mouse MBD2b (SEQ ID NO:2). The underlined amino acids correspond to conserved residues among the DNA binding domains of MBD proteins (Ohki et al. (1999) EMBO J. 18:6653-61). FIG. 5 C shows the results of methylated DNA fractionation using immobilized GST-MBD. The S (supernatant) fraction contains amplified unmethylated SNRPN CpG island DNA. E1 contains amplified methylated SNRPN CpG island DNA that was eluted from the immobilized protein. Fractions E2 and E3 are two additional serial elutions from the same immobilized protein. FIG. 5 D shows the fractionation of PTGS2 DNA, which is unmethylated, in HeLa cells. All of the PTGS2 DNA is recovered in the unbound supernatant fraction S.

FIG. 6 is a flow diagram illustrating the strategy for α-TAP Abscription®-based CpG methylation detection including the binding of a TAP-APC to an amplified fragment of a target CpG island. Steps in the process are indicated by numerals. FIG. 6A illustrates the initial capture of methylated CpG containing DNA. Step 1: Methylated DNA fragments are separated from unmethylated DNA fragments with an immobilized GST-MBD protein. Step 2: Methylated DNA fragments are released by heat treatment or exposure to protease or glutathione. FIG. 6B shows amplification tagging and capture of tagged DNA fragments. Step 3: A target CpG island is tagged with an affinity label such (as biotin (B), as shown) and a single-strand extension during PCR, through the incorporation of a biotinylated primer and a primer containing a non-coding nucleotide between the primer sequence and an anti-TAP sequence (α-TAP). Step 4: The biotinylated amplicon is bound to streptavidin-magnetic beads. Step 5: The APC is bound to the amplicon by hybridization between the TAP sequence and the α-TAP sequence. Abscription® is performed by contacting an RNA polymerase (RNAP) with the bead-immobilized complexes containing APCs.

FIGS. 7A-7C illustrate the interactions between exemplary target-specific amplification primers and anti-TAP (α-TAP) primer/probes. FIG. 7A illustrates the PCR primers used in CpG island amplification and their relative locations in the amplicon. FIGS. 7B and 7C illustrate unfavorable assay outcomes by poorly designed α-TAP primer/probes.

FIG. 8. shows the relative sensitivities of DNA detection by TaqMan® PCR versus the Abscription®/PCR method depicted in FIG. 6. A fragment of the GSTP1 CpG island from unfractionated methylated HeLa DNA was amplified from starting copy numbers/PCR of 9000 to 30. FIG. 8A shows the TagMan® PCR results. TagMan® PCR primers were SEQ ID NO: 3 and SEQ ID NO: 4. Detection of 9000 copies required 28 PCR cycles. FIG. 8 B shows the results of the Abscription®/PCR detection following 29 PCR cycles. Abscription/PCR primers were SEQ ID NO:12 and SEQ ID NO:13. The TAP-APC was made by annealing SEQ ID NO:28 and SEQ ID NO:32. The APC encoded the Abscript GAG. Abscripts were detected using thin layer chromatography (TLC) and UV shadowing. Area refers to the area of the chromatographic peak containing the Abscript.

FIG. 9 shows detection of CY5™ labeled Abscripts using TLC after PCR amplification of the indicated amounts of input DNA followed by 2 hr and 8 hr of Abscription®.

FIG. 10 is a flow diagram showing the strategy for methylated DNA detection using direct incorporation of an APC into amplicons with the use of an APC-primer. Numerals indicate steps in the strategy. Steps 1 and 2 depict the fractionation of methylated DNA using immobilized GST-MBD protein, as illustrated in FIG. 6A. Step 3 shows the relationship between the targeted sequence and the primers that are used to attach an APC to the amplicon. The leftward primer is a conventional PCR primer. The rightward primer has a 3′ priming sequence and a single-stranded APC at the 5′ end. Steps 4 and 5 represent PCR amplification of the target. Step 6 represents the Abscription® step following PCR. The PCR reaction is supplemented with RNA polymerase, initiator and one or more NTPs.

FIG. 11 illustrates the validation of an APC promoter pair for the GAPDH CpG island. FIG. 11A shows the evaluation of background signal due to self-priming by the APC primer C443 and background due to the formation of primer-dimers between C443 (SEQ ID NO:33) and the reverse primer C446 (SEQ ID NO:34). PCR reactions lacking DNA containing C443 alone or a combination of C443 and C446 were performed at a range of annealing temperatures from 56.4° C. to 68.5° C. followed by 1 hr of Abscription®. The production of Abscripts was assayed by TLC-UV shadowing. Only the positive control containing HeLa DNA produced the Abscript GAG. FIG. 11B shows the results of LC-MS detection of Abscripts from the same sample sets.

FIG. 12A-C illustrate the steps in Rapid Thin Layer Chromatography (rTLC)

FIG. 13A illustrates APC-primer pair specificity. FIG. 13A APC-SNRPN F2 and SNRPN R2 were tested for specific amplification of the SNRPN and their ability to synthesize abscripts by Coupled Abscription PCR at a series of increasing stringencies in the absence and presence of HeLa DNA. Samples were analyzed by LC-MS and the total amount of trinucleotide abscript made was measured and plotted as the relative peak area. Abscription signals in the absence of DNA indicate primer dimers or self priming events that activate the APC independently of a DNA target. FIG. 13B. Assays in the presence of DNA indicate the optimum annealing temperature that avoids self priming without unnecessarily sacrificing abortive transcript yield in the presence of DNA. FIG. 13C. The SNRPN APC-primer pairs APC-SNRPN F1-SNRPN R1 (I) and APC-SNRPN F2-SNRPN-R2 (II) were used to amplify 3,000 copies of HeLa DNA for 32 cycles. Samples were fractionated in a 2% w/v agarose gel followed by staining with ethidium bromide. Lane 1 shows the amplification reaction containing DNA. Lane 2 represents the no-template control. Lane 3 shows a no-amplification control containing DNA but lacking Taq DNA polymerase. The expected amplicons contain 301 nucleotides (APC-SNRPN F1-R1) and 117 nucleotides (APC-SNRPN F2-R2).

FIG. 13 illustrates shows results based on abscript detection by rapid Thin Layer Chromatography (rTLC) and Liquid Chromatography-Mass Spectrometry (LC-MS). Rapid TLC had the advantage of allowing rapid processing of multiple samples in parallel. Although the results are qualitative, the imprinting disorders lend themselves to a qualitative yes-no analysis since the samples are expected to contain either all methylated, all unmethylated or a 1:1 ratio of both types of DNA. FIG. 13A shows typical results for PWS (abscript GAG only in the methylated eluted fraction), AS (GAG only in the unmethylated supernatant fraction) and Normal (equal amounts of abscript GAG in both fractions). In all cases visual inspection of rTLC results for the 54 samples led to classifications of the samples in agreement with the more quantitative mass spectrometry assay (FIG. 13B).

FIG. 14 illustrates sensitivity and dynamic range of CAP. FIG. 14A. Calibration curves were made with dilutions of HeLa DNA. The Ct values for detection of 10, 100, 1,000 and 10,000 copies are plotted for TaqMan PCR (open circles, Efficiency: 102%; r2: 0.999). The minimal cycle number at which a plotted DNA copy number can be detected is shown for a series of end-point CAP experiments (filled rectangles, filled and open triangles). The limit of detection at a given CAP cycle number was calculated using curve fitting of the relationship between LC-MS signal and DNA copy number for each titration. The LOD was fixed to a signal intensity of 100 (unfilled squares represent the processed versions of the filled squares). Abscription reactions were for either 3 hr (squares) or for 15 min (triangles). FIG. 14B. HeLa DNA was titrated at the copy numbers indicated on the ordinate. Samples containing 3 to 1,000 molecules were amplified for 28 PCR cycles followed by 30 min of Abscription (circles). Samples containing 1,000 to 100,000 molecules were amplified for 20 PCR cycles followed by 15 min of Abscription (squares). Abscript signals were detected by LC-MS. Primer sequences used in the assay are listed in Table 8.

FIG. 15A-C show detection of methylated DNA from saliva, urine, and FFPE tumor slides. FIGS. 15A and B: Purified normal DNAs from saliva and urine sediments (50 ng) from the same individual were separated into methylated (M) and unmethylated (U) fractions with MethylMagnet. Methylated SNRPN CpG island was detected after 28 cycles of PCR and 15 min Abscription. FIG. 15C. FFPE DNA from normal lung was purified from 2 glass slides (10 mm thickness). SNRPN CpG island DNA was detected after 29 cycles of amplification and 30 min of Abscription.

FIG. 16 shows the region of the SNRPN CpG island that was analyzed with bisulfate sequencing. The MseI generated DNA segment from +234 to +539 nucleotides downstream from the start-site for SNRPN RNA variant 1 is shown. MseI cut HeLa DNA was assayed for methylation of this SNRPN CpG island, which is imprinted and 50% methylated in normal somatic tissues. The average percent methylated DNA determined by MethylMeter was 47.7±2.9%. Unfractionated HeLa DNA was sent out for bisulfite sequencing of the SNRPN segment within the MseI fragment that was probed by MethylMeter. Filled circles are methylated CpG sites (1-24). Five islands were scored as methylated and 5 unmethylated, identical to the MethylMeter results, which took 10× less DNA and far less time and money. Sites in this island that are probed by other common methods are also indicated.

FIG. 17 illustrates workflow according to one embodiment of the invention. FFPE purified DNA is treated with AluI to generate small target fragments that include the priming sites that were used to establish the G-CIMP profile. AluI cleavage is validated by comparing the amplification of cut DNA and an otherwise identical uncut control with a primer pair that cannot amplify AluI cleaved DNA. The fragmented DNA is then separated into an unmethylated fraction and a bead-bound fraction with the use of GST-MBD magnetic beads. The relative amounts of methylated and unmethylated DNAs can be determined by a combination of PCR with promoter primers followed by the production of RNA signals by abscription. Abscription signals are quantified by LC-MS.

FIG. 18 shows quantitation of methylated and unmethylated fractions by MethylMeter. Methylated and unmethylated DNAs are separated by magnetic beads bearing a GST-MBD fusion protein to produce a bead fraction with methylated DNA and a supernatant fraction containing unmethylated DNA (1). The relative amount of either DNA form can be quantified by amplifying specific targets in each fraction.

FIG. 19 shows annealing temperature gradient screen for primer-primer interactions.

FIG. 20 shows the binding capacity of GST-MBD beads as a function of CpG frequency. A 780 ng CIMP+, MseI-PvuII double digested DNA sample was fractionated with 5 βl, 10 μl or 25 μl of GST-MBD beads and the percent methylation was determined for ANKRD43 (241 CpGs), DOCKS (71 CpGs), HFE (27 CpGs) and MAL (26 CpGs). The percent methylation estimations for the low bead inputs were within 80% to 95% of the estimates made at the high bead inputs.

FIG. 20 shows Annealing temperature gradient screen for primer-primer interactions.

FIG. 21 CIMP assignments of characterized validation samples. Twenty samples from MD Anderson Cancer Center were analyzed by the MethylMeter assay and CIMP assignments were made based on the methylation status of 8 markers, A: ANKRD43; D: DOCKS; F: FAS; H: HFE; L: LGALS3; M: MAL; R: RHOF and W: WWTR1. A CIMP plus status was associated with hypermethylation of A, F, H, L, M, R and W, while D was CIMP plus if hypomethylated. A sample was scored as CIMP plus if any combination of 6 markers was CIMP-plus. Numbers in parenthesis after sample IDs represent duplicate assays. MethylMeter results agreed with the MethyLight assignments except for 11-046 (03-16040) where there was a discordance on the status of 3 markers.

### DETAILED DESCRIPTION

### Definitions

It is to be understood that both the foregoing general description and the following detailed description are exemplary and explanatory only and are not restrictive of the invention claimed. As used herein, the use of the singular includes the plural unless specifically stated otherwise. As used herein, “or” means “and/or” unless stated otherwise. As used herein, the terms “comprises,” “comprising”, “includes”, and “including”, or any other variations thereof, are intended to cover a non-exclusive inclusion, such that a process, method, composition, reaction mixture, kit, or apparatus that comprises a list of elements does not include only those elements but may include other elements not expressly listed or inherent to such process, method, composition, reaction mixture, kit, or apparatus. The section headings used herein are for organizational purposes only and are not to be construed as limiting the subject matter described.

Unless specific definitions are provided, the nomenclatures utilized in connection with, and the laboratory procedures and techniques of molecular biology, biochemistry, and organic chemistry described herein are those known in the art. Standard chemical and biological symbols and abbreviations are used interchangeably with the full names represented by such symbols and abbreviations. Thus, for example, the terms “deoxyribonucleic acid” and “DNA” are understood to have identical meaning Standard techniques may be used e.g., for chemical syntheses, chemical analyses, recombinant DNA methodology, and oligonucleotide synthesis. Reactions and purification techniques may be performed e.g., using kits according to manufacturer's specifications, as commonly accomplished in the art or as described herein. The foregoing techniques and procedures may be generally performed according to conventional methods well known in the art and as described in various general or more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)); Ausubel et al. Current Protocols in Molecular Biology (John Wiley & Sons Inc., N.Y. (2003)), the contents of which are incorporated by reference herein in their entirety for any purpose.

“About” as used herein means that a number referred to as “about” comprises the recited number plus or minus 1-10% of that recited number. For example, “about” 50 nucleotides can mean 45-55 nucleotides or as few as 49-51 nucleotides depending on the situation. Whenever it appears herein, a numerical range, such as “45-55”, refers to each integer in the given range; e.g., “45-55 nucleotides” means that the nucleic acid can contain 45 nucleotides, 46 nucleotides, etc., up to and including 55 nucleotides.

“Transcription” as used herein, refers to the enzymatic synthesis of an RNA copy of one strand of DNA (i.e, template) catalyzed by an RNA polymerase (e.g. a DNA-dependent RNA polymerase).

“Abortive transcription” is an RNA polymerase-mediated process that reiteratively synthesizes and terminates the synthesis of oligonucleotides that correspond to at least one portion of a complementary nucleic acid template sequence. Abortive oligonucleotides synthesized in vivo vary in length of nucleotides, and are complementary to a sequence at or near the transcription initiation site.

“Abscription®” is a form of abortive transcription optimized for in vitro analytical use to reiteratively produce short, uniform RNA transcripts or “abscripts” from synthetic or naturally occurring promoter sequences at high frequency in vitro. The term “Abscripts” (capitalized), is used herein to distinguish optimized, synthetic transcripts produced in an Abscription® reaction or assay, from the more general term “abscripts,” which also encompasses short abortive transcripts that are produced during the normal course of transcription as it occurs in nature.

“Reiterative” refers to the repetitive synthesis of multiple identical or substantially identical copies of a sequence of interest.

“Terminator” or “transcription terminator” as used herein, refers to an RNA chain terminating compound, complex or process. A terminator of the invention can, for example, be a nucleotide analog, which can be incorporated into an RNA chain during RNA synthesis to prevent the addition of additional nucleotides to the RNA chain.

“Amplification” as used herein, refers to the process of making identical copies of a polynucleotide, such as a DNA fragment or region. Amplification is generally accomplished by polymerase chain reaction (PCR), but other methods known in the art may be suitable to amplify DNA fragments of the invention.

A “target DNA sequence” or “target DNA” is a DNA sequence of interest for which detection, characterization or quantification is desired. The actual nucleotide sequence of the target DNA may be known or not known. Target DNAs are typically DNAs for which the CpG methylation status is interrogated. A “target DNA fragment” is a segment of DNA containing the target DNA sequence. Target DNA fragments can be produced by any method including e.g., shearing or sonication, but most typically are generated by digestion with one or more restriction endonucleases.

As used herein, a “template” is a polynucleotide from which a complementary oligo- or polynucleotide copy is synthesized.

“Synthesis” generally refers to the process of producing a nucleic acid, via chemical or enzymatic means. Chemical synthesis is typically used for producing single strands of a nucleic acid that can be used and primers and probes. Enzyme mediated “synthesis” encompasses both transcription and replication from a template. Synthesis includes making a single copy or multiple copies of the target. “Multiple copies” means at least 2 copies. A “copy” does not necessarily mean perfect sequence complementarity or identity with the template sequence. For example, copies can include nucleotide analogs, intentional sequence alterations (such as sequence alterations introduced through a primer comprising a sequence that is hybridizable, but not complementary, to the template), and/or sequence errors that occur during synthesis.

The terms “polynucleotide” and “nucleic acid (molecule)” are used interchangeably to refer to polymeric forms of nucleotides of any length. The polynucleotides may contain deoxyribonucleotides, ribonucleotides and/or their analogs. Nucleotides may be modified or unmodified and have any three-dimensional structure, and may perform any function, known or unknown. The term “polynucleotide” includes single-stranded, double-stranded and triple helical molecules. The following are non-limiting embodiments of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers.

“Oligonucleotide” refers to polynucleotides of between 2 and about 100 nucleotides of single- or double-stranded nucleic acid, typically DNA. Oligonucleotides are also known as oligomers or oligos and may be isolated from genes and other biological materials or chemically synthesized by methods known in the art. A “primer” refers to an oligonucleotide containing at least 6 nucleotides, usually single-stranded, that provides a 3′-hydroxyl end for the initiation of enzyme-mediated nucleic acid synthesis. A “polynucleotide probe” or “probe” is a polynucleotide that specifically hybridizes to a complementary polynucleotide sequence. As used herein, “specifically binds” or “specifically hybridizes” refers to the binding, duplexing, or hybridizing of a molecule to another molecule under the given conditions. Thus, a probe or primer “specifically hybridizes” only to its intended target polynucleotide under the given binding conditions, and an antibody “specifically binds” only to its intended target antigen under the given binding conditions. The given conditions are those indicated for binging or hybridization, and include buffer, ionic strength, temperature and other factors that are well within the knowledge of the skilled artisan. The skilled artisan will also be knowledgeable about conditions under which specific binding can be disrupted or dissociated, thus eluting or melting e.g, antibody-antigen, receptor-ligand and primer-target polynucleotide combinations.

“Nucleic acid sequence” refers to the sequence of nucleotide bases in an oligonucleotide or polynucleotide, such as DNA or RNA. For double-strand molecules, a single-strand may be used to represent both strands, the complementary stand being inferred by Watson-Crick base pairing.

The terms “complementary” or “complementarity” are used in reference to a first polynucleotide (which may be an oligonucleotide) which is in “antiparallel association” with a second polynucleotide (which also may be an oligonucleotide). As used herein, the term “antiparallel association” refers to the alignment of two polynucleotides such that individual nucleotides or bases of the two associated polynucleotides are paired substantially in accordance with Watson-Crick base-pairing rules. Complementarity may be “partial,” in which only some of the polynucleotides' bases are matched according to the base pairing rules. Or, there may be “complete” or “total” complementarity between the polynucleotides. Those skilled in the art of nucleic acid technology can determine duplex stability empirically by considering a number of variables, including, for example, the length of the first polynucleotide, which may be an oligonucleotide, the base composition and sequence of the first polynucleotide, and the ionic strength and incidence of mismatched base pairs.

As used herein, the term “hybridization” is used in reference to the base-pairing of complementary nucleic acids, including polynucleotides and oligonucleotides containing 6 or more nucleotides. Hybridization and the strength of hybridization (i.e., the strength of the association between the nucleic acids) is impacted by such factors as the degree of complementary between the nucleic acids, the stringency of the reaction conditions involved, the melting temperature (Tₘ) of the formed hybrid, and the G:C ratio within the duplex nucleic acid. Generally, “hybridization” methods involve annealing a complementary polynucleotide to a target nucleic acid (i.e., the sequence to be detected either by direct or indirect means). The ability of two polynucleotides and/or oligonucleotides containing complementary sequences to locate each other and anneal to one another through base pairing interactions is a well-recognized phenomenon.

A “complex” is an assembly of components. A complex may or may not be stable and may be directly or indirectly detected. For example, as described herein, given certain components of a reaction and the type of product(s) of the reaction, the existence of a complex can be inferred. For example, in the abortive transcription method described herein, a complex is generally an intermediate with respect to a final reiterative synthesis product, such as a final abortive transcription or replication product.

“Methylation” refers to the addition of a methyl group (—CH₃) to a molecule, typically to a nucleotide base in DNA or RNA. “mCpG” refers to a 5′-CG-3′ dinucleotide in which the C is methylated at position 5 (5-methylcytosine or 5-Me C). “CpG islands” are regions of genomic that contain a high frequency of the CpG dinucleotide. CpG Islands are in or near approximately 40% of promoters of mammalian genes and about 70% of human promoters have a high CpG content. See e.g. Fatemi et al. (2005) Nucleic Acids Res. 33:e176. doi:10.1093/nar/gni180. PMID 16314307.

“Promoter” as used herein, refers to a region of DNA that facilitates the transcription of an adjacent gene. Promoters are typically 5′ and proximal to the start site of transcription initiation in a gene, and direct an RNA polymerase and associated transcription factors to the correct location for transcription of a the gene.

“Microarray” and “array,” are used interchangeably to refer to an arrangement of a collection of compounds, samples, or molecules such as oligo- or polynucleotides. Arrays are typically “addressable” such that individual members of the collection have a unique, identifiable position within the arrangement. Arrays can be formed on a solid substrate, such as a glass slide, or on a semi-solid substrate, such as nitrocellulose membrane, or in vessels, such as tubes or microtiter plate wells. A typical arrangement for an array is an 8 row by 12 column configuration, such as with a microtiter plate, however, other arrangements suitable for use in the methods of the present invention will be well within the knowledge of the skilled artisan.

The term “solid support” refers to any solid phase that can be used to immobilize e.g., a capture probe or other oligo- or polynucleotide, a polypeptide, an antibody or other desired molecule or complex. Suitable solid supports will be well known in the art and include, but are not limited to, the walls of wells of a reaction tray, such as a microtiter plate, the walls of test tubes, polystyrene beads, paramagnetic or non-magnetic beads, glass slides, nitrocellulose membranes, nylon membranes, and microparticles such as latex particles. Typical materials for solid supports include, but are not limited to, polyvinyl chloride (PVC), polystytrene, cellulose, agarose, dextran, glass, nylon, latex and derivatives thereof. Further, the solid support may be coated, derivatized or otherwise modified to promote adhesion of the desired molecules and/or to deter non-specific binding or other undesired interactions. The choice of a specific “solid phase” is usually not critical and can be selected by one skilled in the art depending on the methods and assays employed. Conveniently, the solid support can be selected to accommodate various detection methods. For example, 96 or 384 well plates can be used for assays that will be automated, for example by robotic workstations, and/or those that will be detected using, for example, a plate reader. For methods of the present invention that may involve e.g. an autoradiographic detection step utilizing a film-based visualization, the solid support may be a thin membrane, such as a nitrocellulose or nylon membrane, a gel or a thin layer chromatography plate. Suitable methods for immobilizing molecules on solid phases include ionic, hydrophobic, covalent interactions and the like, and combinations thereof. However, the method of immobilization is not typically important, and may involve uncharacterized adsorbtion mechanisms. A “solid support” as used herein, may thus refer to any material which is insoluble, or can be made insoluble by a subsequent reaction. The solid support can be chosen for its intrinsic ability to attract and immobilize a capture reagent. Alternatively, the solid support can retain additional molecules which have the ability to attract and immobilize e.g., a “capture” reagent.

“Antibody” or “antibodies”, as used herein, include naturally occurring species such as polyclonal and monoclonal antibodies as well as any antigen-binding portion, fragment or subunit of a naturally occurring molecule, such as for example Fab, Fab′, and F(ab)₂ fragments of an antibody. Also contemplated for use in the methods of the invention are recombinant, truncated, single chain, chimeric, and hybrid antibodies, including, but not limited to, humanized and primatized antibodies, and other non-naturally occurring antibody forms.

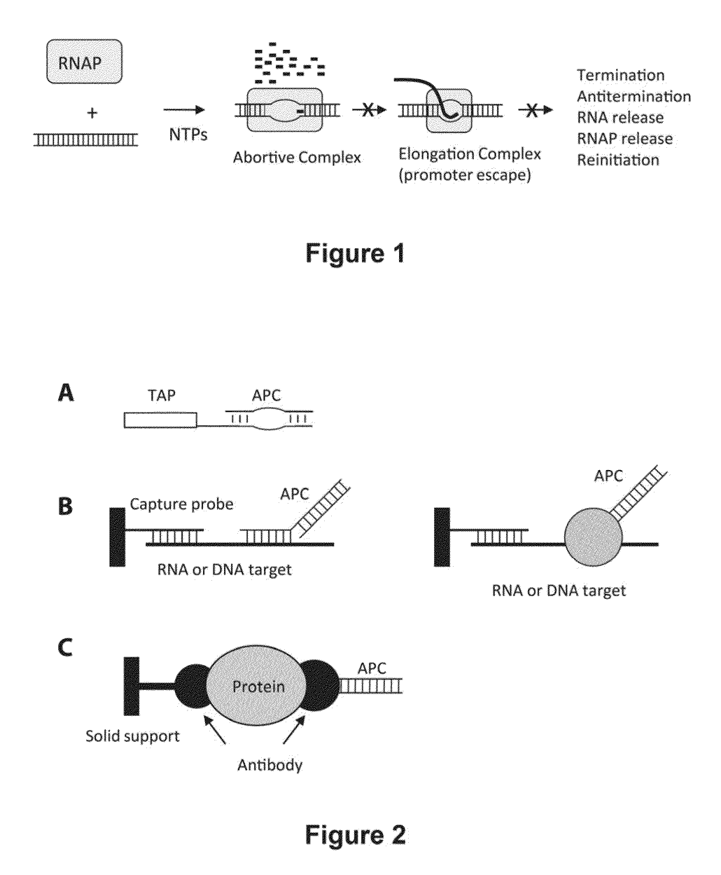

The present invention is based on a molecular detection technology called Abscription® which is in turn based on the natural phenomenon known as abortive transcription (FIG. 1). Abscription® is a robust, isothermal method for detecting and quantifying a wide range of targets including proteins, nucleic acids, SNPs and CpG methylation (U.S. patent application Ser. Nos. 10/602,045, 10/790,766, and 10/488,971; U.S. Pat. Nos. 7,045,319, and 7,226,738). Abscription® occurs during the initiation phase of transcription in which RNA polymerase (RNAP) reiteratively generates short RNAs, or aborted transcripts (Abscripts), while remaining tightly bound to the promoter (Hsu, Biochim. Biophys. Acta (2002) 1577:191-207; Hsu et al. Biochemistry (2003) 42:3777-86; Vo et al. Biochemistry (2003) 42:3798-811; Vo et al. Biochemistry (2003) 42:3787-97; Hsu et al. Biochemistry (2006) 45:8841-54). The sequences of the promoter and the initially transcribed segment have significant effects on the lengths of the predominant Abscripts, as well as their rates of synthesis (Hsu et al. Biochemistry (2006) 45:8841-54.28). Multiple optimal highly abortive promoters, called Abortive Promoter Cassettes (APCs), have been developed and optimized to make Abscripts of different sequences and lengths (between 3 and 12 nt) at extremely high rates. APCs, which include an RNA polymerase binding site and a transcription start site, trap RNA polymerase in the abortive phase and produce only abortive transcripts of a specific sequence. Exemplary APCs are described, e.g., in U.S. Pat. No. 7,473,775, the contents of which is incorporated by reference herein in its entirety.

The generation of short Abscripts is very efficient because the RNAP does not dissociate from the promoter between rounds of truncated RNA synthesis, as it does after producing each full length transcript, and will continue to produce Abscripts at high turnover rates until substrates are depleted. This results in the very rapid production of thousands of Abscripts per APC each minute. Abscription® is a signal amplification, rather than a target amplification process.

The present invention provides simple and sensitive methods for the detection of CpG methylation in DNA via mCpG target site probes that include optimized methyl binding domain (MBD) polypeptides. mCpG target site probes can be coupled directly or indirectly to a signal generator, which produces a detectable signal that can be measured as an indicator of CpG methylation.

In certain embodiments of the invention, signal generation is based on an Abscription® process in which Abortive Promoter Cassettes (APCs) signal generators are bound to target mCpG sites via mCpG target specific probes. RNA polymerase produces uniform, short RNA molecules from synthetic or naturally occurring abortive promoters in APCs as signals (indicators) of the presence of methylated CpGs. In other embodiments of the invention, signal-generating cassettes can produce detectable RNA or DNA signals through PCR or other replication and/or amplification methods.

The methods of the invention offer significant advantages over current CpG methylation detection methods because bisulfite treatment is not required. Thus, the extensive DNA degradation and the reduction of sequence complexity associated with chemical treatment of target DNA can be avoided entirely in certain embodiments of the invention. The methods of the invention are rapid and can typically be performed in a single day. Furthermore, the invention can be adapted to multiplex and automated applications.

Certain Abscription®-based methylation detection assays of the present invention offer the unique capability of coupling a linear, robust signal amplification process (Abscription®) with a target amplification process (e.g. polymerase chain reaction or PCR). This provides for extremely high sensitivity and allows testing to use only small amounts of starting material. In addition, unlike other signal amplification methods, such as horseradish peroxidase or alkaline phosphatase, which generate the same signal molecule from each target in a sample, Abscription® based amplification can be formatted to generate a different signal from each target. These signals, in the form of short oligonucleotides, can then be detected by a variety of methods. Abscription®-based assays require fewer man-hours of labor than other DNA methylation detection assays, reagent cost is very competitive, and instrumentation cost is low. In addition, these assays, by including positive and negative control templates, result in highly specific detection and fewer false positives than other methods for target detection.

### Abscription® Technology

Abscription® technology is based on the observation that prior to the initiation of full-length RNA transcription, a large number of short, abortive transcripts are synthesized by RNA polymerases before full-length RNA transcripts are made. As described below, abortive transcripts are a normal by-product of the transcription process, yet are distinguishable from full-length RNA transcripts (which are the functionally informative product of the transcription process), in both size and in the manner in which they are made.

Transcription Process.

Transcription is a complex and highly regulated process utilized by both eukaryotes and prokaryotes to selectively synthesize RNA transcripts from DNA templates (i.e. genes) (reviewed in Record et al. (1996) Escherichia coli and Salmonella, (Neidhart, ed.; ASM Press, Washington, D.C.); deHaseth et al. (1998) J. Bact. 180:3019-25; Hsu (2002) Biochim. Biophys. Acta. 1577:191-207; Murakami & Darst (2003) Curr. Opin. Struct. Biol. 13:31-39; Young et al. (2002). Cell. 109:417-420). Transcription in a cellular environment includes 5 stages: 1. Preinitiation, during which transcriptional machinery (e.g. RNA polymerase (RNAP) and transcription factors), is recruited to a promoter; 2. Initiation, during which synthesis of RNA begins; 3. Promoter Escape, during which the RNA polymerase leaves the promoter and abortive initiation stops (usually after synthesis of approximately 12-mer RNAs); 4. Elongation, during which RNAP travels processively along the template DNA strand, thereby synthesizing a full-length RNA transcript; and 5. Termination, during which RNA synthesis ceases and RNAP dissociates from the template DNA.

Production of Abortive Transcripts Prior to Full-Length RNA Transcription.

Typically, RNAP fails to escape from the promoter on its first attempt and, instead, engages in multiple abortive cycles of synthesis and release of short RNA products called abortive transcripts. Only when RNAP succeeds in synthesizing an RNA product of a threshold length does RNAP irrevocably break its interactions with promoter DNA, and begin to translocate along the DNA template, processively synthesizing a full-length RNA transcript (see Hsu (2002) Biochim. Biophys. Acta. 1577:191-207; Hsu et al. (2003) Biochemistry 42: 3777-86; Vo et al. (2003) Biochemistry 42:3787-97; Vo et al. (2003) Biochemistry: 42:3798-11). Prior to promoter escape in (stage 3, above), RNAP remains bound to template DNA at or near the promoter region, thereby allowing multiple rounds of abortive synthesis in a short time.

Abscription® Technology.

Abscription® technology exploits the natural phenomenon of abortive RNA synthesis to produce large numbers of detectable abortive transcripts (Abscripts). Abscription® is an isothermal, robust, linear signal generation system based on abortive transcription. In an Abscription® method, Abortive Promoter Cassettes (APCs) are bound to target molecules via Target Site Probes (TSPs). An RNA polymerase, such as E. coli RNA polymerase, then uses the APC as a template for generating large numbers of signals per target in the form of short, uniform RNA molecules or Abscripts (abortive transcripts).

Abscription® detection methods have three basic steps that can be adapted to detect a wide variety of molecules of interest (i.e. targets). First, an APC is localized to a target molecule of interest through a Target Site Probe (TSP). Second, Abscripts are synthesized from the localized APCs. Finally, Abscripts are detected as a means of target detection and may be quantified as an indication of the amount of a target molecule present. The process is very efficient because the RNAP does not move away or dissociate from the promoter between rounds of abortive RNA synthesis, as it does after producing each full-length transcript. Furthermore, only uniform, short RNA signals are synthesized, which can be produced more quickly and with less effort than longer oligo- and polynucleotides.

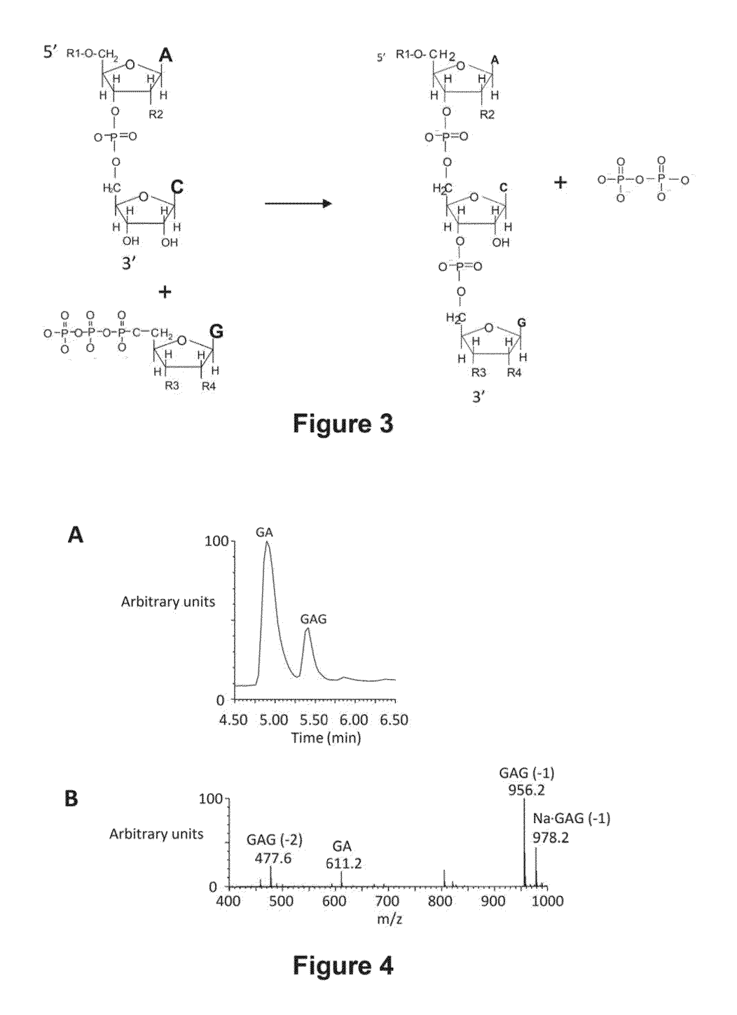

Although the factors and conditions required for promoter escape (and hence the end of abortive synthesis), are incompletely understood, sufficient knowledge is available to create a synthetic environment that favors abortive transcript synthesis and precludes full-length RNA production. In one embodiment, Abscription® is controlled at the synthesis stage to produce Abscripts that are initiated with a defined dinucleotide initiator and then terminated after the addition of one or more NTPs as illustrated in the nonlimiting example shown in FIG. 3. Abscript length can be limited to as short as 3 nucleotides (nt) with the use of chain terminating NTPs (e.g., 3′-O-Me-NTPs) or by omitting one or more NTPs from the reaction.

In other embodiments, Abscript length is controlled at the promoter/template stage, by providing synthetic templates that have a discrete, limited number of nucleotides available for transcription before a stop signal is reached. The uniformity of Abscript production from a single APC in a single Abscription® reaction results in Abscript signals that are directly proportional to the amount of target present. Thus Abscription® is both a qualitative and a quantitative system for measuring a target, such as mCpG.

Abortive promoters can be incorporated into DNA targets through a target amplification processes or formed on single-stranded DNA targets by hybridization of a second strand. More generally however, Abscription® can be used to detect a wide variety of target molecules by binding APCs to those targets (FIG. 2). For detection of protein, RNA or DNA, APCs are connected to a Target Attachment Probe (TAP) which will bind to the molecular target (FIG. 2A). For detection of DNA and RNA, TAPs include oligonucleotides or proteins that bind specifically to the nucleic acid target (FIG. 2B). For protein detection, APCs can be attached to anything that binds to the protein target. Several assays have been developed which employ two antibodies directed to the same targe, similar to ELISA, one for capture of the target and one for attachment of the APC (FIG. 2C).

### Trinucleotide Synthesis

Trinucleotide Abscripts can be made exclusively with the inclusion of chain terminator NTPs or by omitting one or more NTP. Abscripts can be labeled for detection or capture by incorporating modified dinucleotides (Dissinger & Hanna, J. Biol. Chem. (1990) 265:7662-8: Dissinger & Hanna, J. Mol. Biol. (1991) 219:11-25; Hanna, Meth. Enzymol., (1989) 180:383-409; Hanna et al. Biochemistry (1989) 28:5814-20; Hanna & Meares, Proc. Natl. Acad. Sci. USA, (1983) 80:4238-42; Hanna & Meares, Biochemistry (1983) 22:3546-51.29-34), or NTPs (Hanna, et al. Nucleic Acids Res. (1999) 27:1369-76; He et al, Nucleic Acids Res (1995) 23:1231-8) during Abscription®. “Label” as used herein refers to a moiety, the presence of which on a molecule, can be detected and distinguished either directly or indirectly. Labels typically are more readily detected, with higher efficiently and/or specificity than detecting a native, unlabeled species. Label suitable for use in the methods of the present invention include fluorescent groups, affinity tags (such as biotin), and charge or mass modified nucleotides.

In one embodiment, the assays described herein involve the production of different trinucleotide Abscripts that differ by molecular weight or mobility. Trinucleotides are made by RNAP at rates of 1000 to 2000 per minute on APCs by joining a dinucleotide initiator and a nucleoside triphosphate. Trinucleotide Abscripts can be detected without labeling by rapid TLC and UV shadowing or mass spectrometry. Alternatively, Abscripts can be detected through a label (e.g. a fluorescent moiety) incorporated into a dinucleotide initiator.

The present invention provides methods for detecting a polynucleotide in a sample by contacting a sample containing the polynucleotide with a primer pair that specifically hybridizes to and amplifies a target sequence of the polynucleotide. The primer pair includes a first primer that is complementary to the polynucleotide, flanks the target sequence, and contains a 5′ capture tag. The second primer has three regions: a 3′ sequence complementary to the polynucleotide that flanks the target sequence on the opposite side of the target sequence from the first primer; a 5′ α-TAP sequence that is used to attach the APC following amplification; and a non-natural nucleotide between the 3′ and 5′ sequences, that is typically an etheno-deoxyadenosine.

The target sequence of the polynucleotide is then amplified (e.g. by polymerase chain reaction) using the first and second primers and the amplified target sequence is captured on a solid support containing a molecule that binds the 5′ capture tag. Any available PCR technique or suitable nucleic acid amplification method can be employed for this step, such as PCR methods that use thermostable DNA polymerase and/or RNA polymerase enzymes. For example the capture tag can be biotin and the solid support can be streptavidin beads, such as magnetic beads. The captured amplicons can then be washed to remove unbound primers and, if desired, eluted from the solid support.

For detection of the target polynucleotide sequence, a probe is hybridized to the amplicon. This probe includes a 5′ TAP sequence complementary to the α-TAP sequence. Due to the inclusion of the non-natural nucleotide in the second PCR primer, the α-TAP sequence is not copied during PCR and remains single-stranded during the amplification, thereby allowing the TAP sequence of the probe to hybridize without denaturing the amplified target. Etheno-deoxyadenosine can be used as the non-natural nucleotide, but the skilled artisan will be aware of additional suitable non-natural nucleotides (e.g. nucleotide analogs) that terminate replication and can thus be substituted for etheno-deoxyadenosine. The probe also includes an APC, which provides the template for synthesizing Abscripts using Abscription® methods as described above. Finally, the Abscripts are detected by any suitable method, particularly the methods described herein for Abscript detection, such as mass spectrometry, capillary electrophoresis or thin layer chromatography. Typically, the Abscripts will have a length of from 3 to 20 nucleotides and may be labeled by incorporating a detectably-labeled (e.g. fluorescent) nucleotide during Abscription®.

In other embodiments of the invention, the TAP/α-TAP step can be eliminated by including an APC sequence at the 5′ end of the second amplification primer and omitting the non-naturally occurring nucleotide. In such embodiments, a double-strand APC is generated during amplification adjacent to the amplified target sequence, which will direct Abscription® upon addition of RNAP and nucleotides. If these Abscription® reagents are present during amplification, Abscription® and amplification can be performed simultaneously in the same tube.

These methods of the invention can be adapted for multiplexing (i.e., detection of a plurality of polynucleotides simultaneously) by including primer pairs specific to each polynucleotide in the reaction. According to this embodiment of the invention, each primer pair is designed to specifically hybridize to and amplify a unique target sequence of a polynucleotide. The α-TAP sequence for each primer pair is also unique, thereby acting as an identifier for the target sequence. By hybridizing a complementary TAP sequence that includes a unique identifying APC following PCR amplification, the presence of each polynucleotide can be interrogated based on the Abscripts produced in the multiplex reaction. Thus a unique APC is attached to each amplified target polynucleotide and the distinguishable Abscript signal produced from the APC can be detected and measured as an indication of the presence of the target polynucleotide. For example, each APC can be designed to generate an Abscript distinguishable on the basis of molecule weight or nucleotide sequence. In a single reaction, 5, 10, 20 or more unique target sequences can be detected.

The present invention also provides methods for determining the methylation status of CpG islands without the use of deamination with bisulfite. Such method combines target amplification with a linear signal amplification process, Abscription®, making it extremely sensitive.

In certain embodiments of the invention, the target polynucleotide(s) is a methylated CpG island or a plurality of CpG islands (i.e. multiplexing). In these embodiments, genomic DNA containing methylated CpG islands is first cleaved using a predetermined restriction enzyme that does not cut the island or any of the islands in a multiplex assay. Methylated DNA fragments are then captured from the genomic DNA using an immobilized MBD reagent and the capture fragments interrogated for specific CpG sequences of interest. According to one method of the invention, the process begins by isolation of methylated DNA from fragmented genomic DNA using a methylated DNA enrichment process. In one aspect of the invention, the enrichment method uses a glutathione-S-transferase fusion protein which contains the methyl binding domain from mouse MBD2, which is highly specific for methylated DNA. Methylated DNA bound to MBD2-fusion protein is captured rapidly by glutathione magnetic beads and eluted directly into buffer for amplification by the polymerase chain reaction. CpG islands of interest are amplified using a pair of modified PCR primers. The first contains a biotin group for subsequent capture of the targeted CpG island to streptavidin magnetic beads. The second primer contains an island-specific sequence that “marks” the amplicon for attachment of a specific APC. Once amplified, islands are captured to streptavidin beads; unique APCs are attached by hybridization; and Abscription® is initiated. Each CpG island thereby generates a different Abscript; therefore multiple CpG islands can be interrogated in each reaction.

Alternatively, an APC sequence can be incorporated into the second amplification primer, and an APC duplex generated during amplification. This approach allows amplification and Abscription® to be performed at the same time by including RNAP and nucleotides in the amplification reaction. Because this method couples target amplification with signal amplification, less starting DNA is required than with most methylated DNA detection methods, and less than 2 ng of genomic DNA is sufficient for the initial step of isolating methylated DNA. The entire assay is very rapid, requiring less operator time than most competing assays. The assay can be used as described with magnetic beads and can also be formatted for high throughput screening in a microtiter plate format.

### EXAMPLES

### Example 1

### Abscription® Methods

Abscription® has been previously described; see e.g. U.S. patent application Ser. No. 09/984,664 (filed Oct. 30, 2001) now U.S. Pat. No. 7,045,319; Ser. No. 10/425,037 (filed Apr. 29, 2003); Ser. No. 10/600,581 (filed Jun. 23, 2003); Ser. No. 10/602,045 (filed Jun. 24, 2003); Ser. No. 10/607,136 (filed Jun. 27, 2003), now U.S. Pat. No. 7,226,738; Ser. No. 10/686,713 (filed Oct. 17, 2003); Ser. No. 10/976,240 (filed Oct. 29, 2004); Ser. No. 10/790,766 (filed Mar. 3, 2004); Ser. No. 10/488,971 (filed Oct. 18, 2004); and Ser. No. 10/551,775 (filed Sep. 14, 2006) the contents of each of which are incorporated by reference herein in their entirety.

### Example 2

### Mass Spectrometry Detection of Abscripts

Trinucleotide Abscripts are detected by mass spectrometry following their fractionation from dinucleotide initiators by HPLC. The output of the fractionation is plotted as total ion count verses chromatographic retention time as illustrated in FIG. 4A. The chromatographic profile for any ion can be similarly plotted. The contributions of particular m/z species at a specific retention time can be summed to give the amount of Abscript as the area under the chromatographic peak. FIG. 4B shows the ion spectrum associated with the Abscript GAG (retention time of 5.4 min). The yield of GAG would be the sum of species with m/z values of 477.6, 956.1 and 978.2. These species account for doubly charged, singly charged and the sodium adduct respectively.

### Example 3

### Preparation of GST-MBD Protein

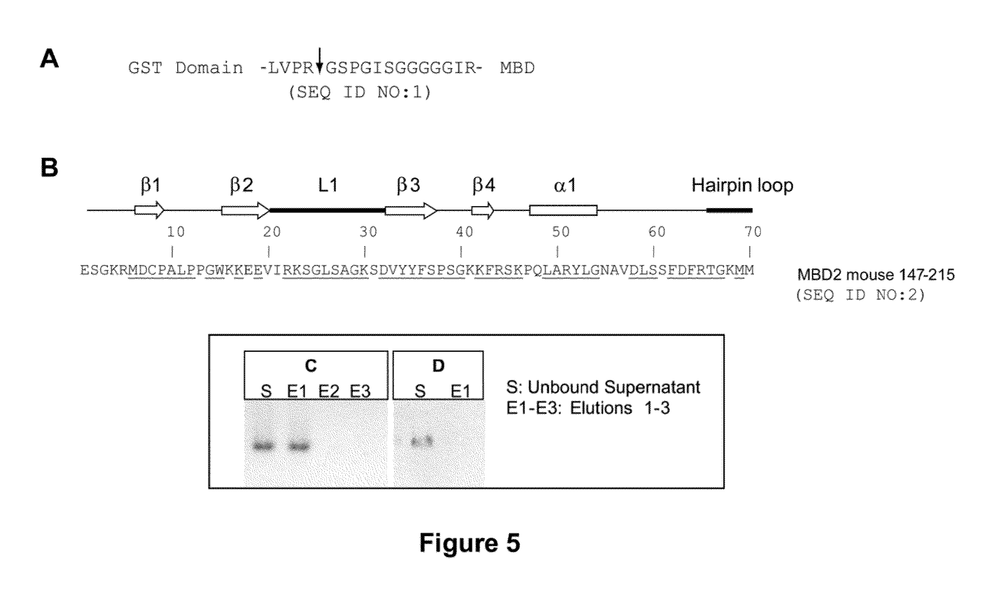

A GST fusion protein that contains the methyl binding domain (MBD) from mouse MBD2 was constructed as illustrated in FIG. 5. The codons for the MBD domain were optimized for expression in E. coli. The construct contains a thrombin cleavage site between the GST and MBD domains. The GST protein also contains four surface cysteine residues that were used for attachment of APCs or Biotin. The details of the GST-MBD protein are provided in U.S. Patent Application No. 61/053,648, filed May 15, 2008 the contents of which are incorporated by reference herein, and in particular, Examples 2-11 describing the preparation and use of MBD fusion proteins.

The GST domain allows the fusion protein, or its complexes with methylated DNA, to be isolated on Glutathione resins or beads and eluted with glutathione, or to be captured or detected with antibodies that recognize GST.

MBD from the MBD2b protein was chosen for final constructs because MBD2b has the highest affinity among the known methyl CpG binding proteins for Me-CpG sites and the lowest cross reactivity with unmethylated CpGs. It has between a 25 to 100 fold higher affinity for Me-CpG sites than does MeCP2, and a 9.7 to 43 fold higher preference for methylated DNA than does MeCP2 (Fraga et al. (2003) Nucleic Acids Res., 31:1765-74). Additionally, there are no sequence context effects on MBD2 CpG recognition, as there are for MeCP2, which requires a run of 4 A-Ts near a CpG site. Therefore a greater number of mCpG sites are recognized by MDB2 than by MeCP2.

### Example 4

### Immobilized GST-MBD2 Retains High Specificity for Methylated DNA Even With 2 ng or Less of Input DNA

The GST-MBD protein was attached to glutathione magnetic beads and used to isolate varying amounts of methylated DNA to determine the minimum starting DNA sample size that could be recovered with high specificity. HeLa genomic DNA or HeLa DNA artificially methylated with SssI methylase was incubated with GST-MBD magnetic beads for 1 hour at 22° C. with horizontal rotary mixing at 1000 rpm. Bound DNA was eluted from the beads after removal of the supernatant containing unbound DNA, by incubation at 80° C. for 10 min with horizontal rotary mixing at 1000 rpm. Eluted DNA samples were tested for the presence of PTGS2 (GenBank GI:34576917) DNA by qPCR using the primer pair 5′-ggtacgaaaaggcggaaaga-3′ (SEQ ID NO:6) and 5′-tgtgggaaagctggaatatc-3′ (SEQ ID NO:7) with SYBR® Green dye for detection. PTGS2 is unmethylated in HeLa and was expected to remain in the supernatant of the binding reaction. A recovery of 94% of a 2 ng genomic DNA input of artificially methylated DNA was observed in the eluted fraction while 100% of the unmodified PTGS2 DNA from HeLa was recovered in the supernatant fraction as expected. At an input of 1 ng of methylated HeLa DNA, 73% of PTGS2 DNA was bound and recovered with heat elution. No binding of the unmethylated version from unmodified HeLa was detected. The lowest DNA amount (2 ng) which could be visualized by agarose gel electrophoresis staining corresponds to approximately 300 cells. Even less DNA can be isolated and detected using Abscription® for amplicon detection.

### Example 5

### Abscription® Based CpG Methylation Assay

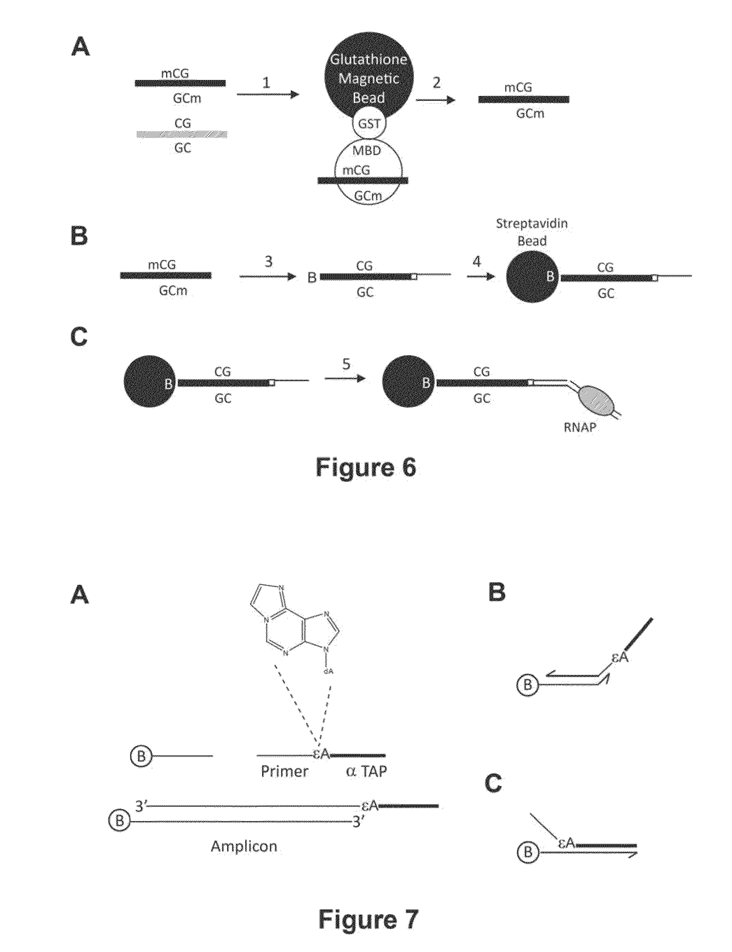

FIG. 6 illustrates an overall protocol for determining the methylation status of multiple CpG islands. Briefly, fragmented methylated DNA from a genomic DNA sample is isolated, followed by amplification of specific CpG islands whose methylation status is under investigation. For the amplification, one primer contains an affinity tag, such as biotin, which allows retrieval and immobilization of the island. The second primer contains a sequence for attachment of the Abortive Promoter Cassette.

This method permits the amplification and isolation of as many CpG islands in a sample as compatible PCR primers can be designed. Because the DNA is not deaminated with bisulfite treatment, which results in the conversion of “C”s to “dU”, the problematic issues for high level multiplexing associated with the loss of sequence heterogeneity caused by deamination, are avoided. Since the primer sites are not rigidly limited to specific sequences in the target, there is sufficient flexibility in primer placement to allow the design of multiple compatible primer sets (Henegariu et al. Biotechniques (1997) 23:504-11; Onishi et al. J. Agric. Food Chem. (2005) 53:9713). A different APC is attached to each target CpG island, thereby generating a different Abscript signal for each. Thus, simultaneous detection of multiple CpG islands from a single sample can be achieved.

Briefly, native genomic DNA is fragmented and then bound to glutathione beads containing the Glutathione-S-Transferase (GST)—Methyl Binding Domain (MBD) fusion protein described above in EXAMPLE 3. Only methylated DNA binds (FIG. 6A, Step 1). After washing, the methylated DNA is eluted from the beads (FIG. 6A, Step 2) and islands to be interrogated are amplified by PCR (FIG. 6B. Step 3). One of the primers contains a capture tag (e.g. biotin) at the 5′ end (FIG. 7A). The second primer contains 2 regions. The first is a polynucleotide sequence complementary to the target DNA. The second region is an arbitrary sequence dissimilar to the target DNA or other islands (FIG. 7A). This second sequence is complementary to a Target Attachment Probe (TAP) sequence which is linked to an Abortive Promoter Cassette (APC). The complement to the TAP sequence is called an anti-TAP sequence (α-TAP). The α-TAP sequence remains single-stranded during the PCR amplification of the target DNA due to the inclusion of a non-natural nucleotide at the junction between the primer sequence that hybridizes to the target and the α-TAP sequence (the EA nucleotide in FIG. 7A). After amplification, the islands are immobilized, for example on streptavidin beads (FIG. 6B, Step 4), and remaining genomic DNA and primers are washed away. The TAP-APC polynucleotide is then contacted with the amplified target DNA and hybridizes to the single-stranded anti-TAP sequence (FIG. 6C, Step 5). Free TAP-APC is washed away, and Abscription® reagents are added to generate Abscript signals (FIG. 6C, Step 5). Further details of the method are given below.

### Step 1: Cutting of Genomic DNA

The initial digestion step was designed to generate fragments that contain CpG islands or large portions thereof. The restriction sites were chosen to fall outside of the region to be analyzed and are neither methylation dependent nor methylation sensitive. Restriction enzymes with 4 base recognition sequences were used. Table 1 lists the fragment sizes generated by MseI or DdeI for a sample of 6 CpG islands reported to be differentially methylated. Up to 3 μg of genomic DNA routinely were digested with 20 units of MseI (NEB, Beverly, Mass.) in the vendor's restriction buffer (NEB buffer 4). Cleavage reactions were incubated for at least 8 hr at 37° C. The extent of cleavage was measured by performing PCR on a sample of the digest using a primer that contains the MseI sequence. Positive controls are genomic DNA untreated with MseI and an amplification reaction of the MseI treated DNA using primers that are unaffected by MseI digestion.

The purpose of the digestion is to unlink the target islands from neighboring CpG sequences that might be normally methylated and thereby cause the transfer of an unmethylated island to the methylated DNA fraction. In most cases either MseI or Dde I produced a single fragment that accounts for the bulk of the island sequence without including many neighboring sequences. The exception was the excessive cleavage of MGMT with DdeI. In this case, the alternative enzyme produced satisfactory results. In cases where a CpG island is fragmented into several fragments, each segment can be analyzed with its own set of primers.

TABLE 1
CpG Island Cleavage patterns

| | Restriction | Recognition | |
|---|---|---|---|
| CpG Island | Enzyme | Sequence | Fragment(s) generated (nt) |
| APC | MseI | TTAA | 520, 164 |
| (GI: 224589817) | DdeI | CTNAG | 820 |
| CCNA1 | MseI | TTAA | 1168 |
| (GI: 224589804) | DdeI | CTNAG | 523, 231, 177 |
| GSTP1 | MseI | TTAA | 1936 |
| (GI: 34576917) | DdeI | CTNAG | 261, 131 |
| MGMT | MseI | TTAA | 1967 |
| (GI: 34556) | DdeI | CTNAG | Excessively fragmented |
| RARB | MseI | TTAA | 612, 163 |
| (GI: 35881) | DdeI | CTNAG | 625 |
| PTGS2 | MseI | TTAA | 516, 417 |
| (GI: 211904109) | DdeI | CTNAG | 464, 229, 87, 37 |

### Step 2: Isolation and Recovery of Methylated DNA

The methylated DNA capture step was formatted for magnetic beads bearing glutathione. The GST-MBD protein was preloaded onto the beads suspended in the DNA sample after removing excess GST-MBD protein. DNA binding was performed for 1 hr at room temperature (22-24° C.) with mixing to maintain the beads in a suspended state (Eppendorf Thermomixer, 1000 rpm). DNA samples typically contained between 1 ng to 50 ng of genomic DNA in a 50 μl volume of binding buffer. At the completion of the binding step the beads were pelleted to the side of the tube with a rare-earth magnet and the supernatant containing unmethylated DNA was removed. The beads were washed twice with, 400 μl of a wash buffer containing the same NaCl concentration as the binding buffer (160 mM). Each wash was incubated for 5 min at room temperature with mixing (1000 rpm). A final wash was performed with TE buffer (10 mM Tris pH 8, 1 mM EDTA). The beads were suspended in 400 μl TE and immediately pelleted with the magnet. The TE wash buffer was discarded and the beads were suspended in 50 μl of elution buffer (10 mM Tris pH 8, 1 mM EDTA).

Methylated DNA can be eluted from the beads with several alternative methods. Beads in TE buffer can be incubated for 10 min at 80° C. with mixing (1000 rpm). The beads are pelleted with the magnet and the eluted DNA is recovered. In an alternative method the beads are suspended in elution buffer containing 0.1% SDS. Complete elution of bound methylated DNA was achieved with a single 20 min incubation at 50° C. The eluted DNA is ready for PCR without further processing provided a nonionic detergent such as Tween-20 is included in the PCR buffer (see Goldenberger et al. PCR Methods Appl. (1995) 4:368-70). Elution can also be performed with exposure of the beads for 10 min to elution buffer containing a minimum of 20 mM reduced glutathione at pH 8. FIG. 5C shows the results of the fractionation of the SNRPN CpG island of HeLa DNA. Methylated DNA was released with heat treatment. SNRPN is an imprinted gene. One copy is fully methylated and the other copy is normally unmethylated. As expected half of the SNRPN copies were found in the supernatant (FIG. 5C fraction S, unmethylated fraction) and half of the copies were eluted from the beads in the first elution (FIG. 5C, fraction E1, methylated fraction). All of the methylated DNA was extracted in the first elution. Two serial elutions after the first elution (E2 and E3) did not contain SNRPN DNA. The CpG island of the PTGS2 gene (unmethylated in HeLa) was used as a negative control (FIG. 5D). All of the PTGS2 DNA appeared in the supernatant fraction.

Step 3: PCR Amplification with Tagged Primers

CpG island segments were amplified and labeled with a biotin affinity tag and a single-stranded oligonucleotide sequence that is used for attachment of an abortive promoter cassette (APC). PCR reactions contained 1× Hot Start Taq buffer (Fermentas) 0.8 mM dNTPs (0.2 mM each), 2 mM MgCl₂, and 5% (v/v) DMSO. The biotinylated primer and the α-TAP primer were present at 1 μM each. Amplifications were performed with 2 units/20 μl reaction of TrueStart™ hot start Taq DNA polymerase (Fermentas). One unit equals the incorporation of 10 nmol of dNTPs in 30 min at 74° C. The cycling conditions were 95° C. for 1 min, followed by up to 32 cycles of 95° C. for 30 sec, 62° C. for 30 sec, and 72° C. for 30 sec. A final elongation step was at 72° C. for 5 min. Completed reactions were held at 4° C. FIGS. 5C and 5D show detection of the fractionated, amplified DNAs by agarose gel electrophoresis.

### α-TAP Primer Sequence Design

Primers were optimized for high signal intensity by minimizing interactions among the three oligonucleotide primer/α-TAP sequences that are present in the PCR reaction. The biotinylated PCR primer and the 3′ end of the α-TAP were used to prime DNA synthesis during the amplification and were designed using primer software (Oligo Explorer 1.2) to minimize primer:primer interactions and the formation of primer hairpin loops. The α-TAP at the 5′ end of the second primer was designed to be incorporated into the amplicon and remain single-stranded due to the presence of a non-coding nucleotide, ethenoA (εA), which separates the primer sequence from the α-TAP. Most DNA polymerases including Taq polymerase cannot incorporate a dNTP opposite εA and terminate synthesis (FIG. 7) (see Patel et al. J. Biol. Chem. (2001) 276:5044-51). The nucleotide analog prevents the α-TAP sequence from being copied during PCR. Potential interactions between the α-TAP sequence and either primer sequence are minimized to avoid inhibition of PCR and false positive results from nonspecific immobilization of a fully single-stranded α-TAP.

Primer design went through 3 steps. First the priming sequences were optimized within the following criteria: 1) a primer length between 16 to 20 nt; 2) a Tₘ close to 60° C.; 3) primer-primer interactions with base paired 3′ ends were eliminated; 4) other primer-primer interactions must have a Tₘ of <16° C.; and 5) hairpin structures with Tₘ>8° C. were rejected. Primer pairs for 6 CpG islands were successfully developed using these criteria.

The second step in primer optimization was to eliminate hairpin structures in the α-TAP-primer oligonucleotide that might interfere with PCR or attachment of a TAP-APC. A collection of 6 α-TAP candidate sequences were designed based on RNA phage MS2, fr and Q sequences. Each candidate α-TAP was tested in silico and those that formed hairpins with a Tₘ>26° C. under our TAP-annealing conditions were modified to eliminate the hairpin and retested (Zuker, Nucleic Acids Res. (2003) 31:3406-15). The only limitation in modifying α-TAP sequences was that they do not acquire significant complementarity with the priming sequences. Potential hairpin interactions between an α-TAP and a linked primer sequence were tested in silico and if necessary the α-TAP sequence was changed to minimize hairpin stability and/or prevent primer extension from a hairpin. An exemplary primer-α-TAP oligonucleotide (SEQ ID NO:42) has a hairpin structure with a Tₘ of 41° C. under PCR conditions used, but amplified CDKN2A DNA as efficiently as the primer lacking the α-TAP extension in combination with reverse primer SEQ ID NO:43. Finally, interactions between the α-TAP and the biotinylated primer were tested in primer design software using a CpG island sequence file with the α-TAP appended at the end of the file and choosing it as a primer along with the biotinylated primer sequence. α-TAP and reverse primer pairs were developed for CpG islands associated with the α-TAP appended at the end of the file and choosing it as a primer along with the biotinylated primer sequence. α-TAP and reverse primer pairs were developed for CpG islands as listed below in Table 2.

TABLE 2
α-TAP and Reverse Primer Pairs

| CpG Island | α-TAP primer | Reverse Primer |
|---|---|---|
| DAPK1 | 5′-cacaggtcaaaggtcataaaaatg[εA] | 5′-Biotin-gtcctcctcacactccg-3′ |
| (SEQ ID NO: 44) | TTtcccataccaagcaccgt-3′ | (SEQ ID NO: 9) |
| | (SEQ ID NO: 8) | |
| GAPDH | 5′-caccgtcgaatctctcc[εA]ccgtgtgc | 5′-Biotin-gtgcctttcattccatccag |
| (SEQ ID NO: 45) | ccaagacc-3′ | cc-3′ |
| | (SEQ ID NO: 10) | (SEQ ID NO: 11) |
| GSTP1 | 5′-ccaagaagccacacgaca[εA]gcggg | 5′-Biotin-actcactggtggcgaagact-3′ |
| (SEQ ID NO: 47) | accctccagaa-3′ | (SEQ NO ID: 13) |
| | (SEQ ID NO: 12) | |
| MGMT | 5′-cctccatcccaaagtA[εA]cctctgc | 5′-Biotin-ccgatggcctagacactg-3′ |
| (SEQ ID NO: 46) | tccctccgaa-3′ | (SEQ ID NO: 15) |
| | (SEQ ID NO: 14) | |
| PTGS2 | 5′-gaaaggactacaaaggacaga[εA]gg | 5′-Biotin-tgtgggaaagctggaatatc-3′ |
| (SEQ ID NO: 48) | tacgaaaaggcggaaaga-3′ | (SEQ ID NO: 17) |
| | (SEQ ID NO: 16) | |
| SNRPN | 5′-cgaaaatgcatctgagtagc[εA]acctc | 5′-Biotin-ggtatcctgtccgctcgca-3′. |
| (SEQ ID NO: 49) | cgcctaaaatccctatg-3″ | (SEQ ID NO: 19) |
| | (SEQ ID NO: 18) | |

### TAP-APC Design.

TAP-APCs were made by hybridizing TAP-APC non-template strands to complementary APC template strands. The APC portions were double-stranded and the TAP segments were a single strands extending from the non-template strands. TAP-APCs were designed so that a collection of APCs either encoded the same abscript for use in single-plex reactions, or a collection of TAP-APCs each encoded a different abscript allowing for multiplex detection. Single-stranded single-plex TAP-APCs were designed for the α-TAPs as indicated below in Table 3.

TABLE 3
Single-Plex TAP APCs

| CpG | |
|---|---|
| Island | TAP APC |
| DAPK1 | 5′-catttttatgacctttgacctgtggctgttgacacagaat |
| (SEQ ID | aaacgctcaatgtacaatgggatggagaggtgctttagta gt |
| NO: 44) | gtt-3′ (SEQ ID NO: 26) |
| GAPDH | 5′-ggagagattcgacggtgctgttgacacagaataaacgctc |
| (SEQ ID | aatgtacaatgggatggagaggtgctttagtagtgtt-3′ |
| NO: 45) | (SEQ ID NO: 27) |
| GSTP1 | 5′-gtcgtgtggcttcttgggctgttgacacagaataaacgct |
| (SEQ ID | caatgtacaatgggatggagaggtgctttagtagtgtt-3′ |
| NO: 47) | (SEQ ID NO: 28) |
| MGMT | 5′-tactttgggatggagggctgttgacacagaataaacgctc |
| (SEQ ID | aatgtacaatgggatggagaggtgctttagtagtgtt-3′ |
| NO: 46) | (SEQ ID NO: 29) |
| PTGS2 | 5′-ctgtcctttgtagtcctttcggctgttgacacagaataaa |
| (SEQ ID | cgctcaatgtacaatgggatggagaggtgctttagtagtgtt- |
| NO: 48) | 3′ |
| | (SEQ ID NO: 30) |
| SNRPN | 5′-gctactcagatgcattttcggctgttgacacagaataaac |
| (SEQ ID | gctcaatgtacaatgggatggagaggtgctttagtagtgtt- |
| NO: 49) | 3′ |
| | (SEQ ID NO: 31) |

The single-stranded single-plex TAP-APCs were annealed to a common template strand encoding the same abscript (SEQ ID NO:32).

Multiplex-compatible TAP-APCs were each paired with their own template strand encoding a unique abscript. A collection of multiplex TAP-APCs (Table 4) could be used together to detect the CpG islands listed in Table 4.

TABLE 4
Multiplex-Compatible TSP-APCs

| CpG | |
|---|---|
| Island | TAP APC |
| DAPK1 | 5′-catttttatgacctttgacctgtgaaatttatgtttgac |
| (SEQ ID | agatcttacaatgcatgctataataccactaacggtgcttta |
| NO: 44) | aaattccg-3′ (SEQ ID NO: 20) |
| | 5′-cggaattttaaagcaccgttagtggtattatagcatgca |
| | ttgtaagatctgtcaaacataaattt-3′ |
| | (SEQ ID NO: 21) |
| GSTP1 | 5′-gtcgtgtggcttcttggaaatttatgtttgacagatctt |
| (SEQ ID | acaatgcatgctataataccactgaaggtgatataaaattcc |
| NO: 47) | g-3′ (SEQ ID NO: 22) |
| | 5′-cggaattttatatcaccuucagtggtattatagcatgca |
| | ttgtaagatctgtcaaacataaattt-3′ |
| | (SEQ ID NO: 23) |
| MGMT | 5′-tactttgggatggagggctgctggaggcgggtataattt |
| (SEQ ID | agccagcaccgaatagttacggtcg-3′ |
| NO: 46) | (SEQ ID NO: 24) |
| | 5′-cgaccgtaactattcggtgcttagggcagcgcccccgcc |
| | tccacgagc-3′ (SEQ ID NO: 25) |

### Step 4 and 5. Attachment of Amplicons to Streptavidin Beads and Binding of TAP-APCs

Biotinylated amplicons were bound to streptavidin beads to remove free probes and unbound APCs in succeeding steps of the assay.

In optimization experiments, complete binding of amplicons to the streptavidin magnetic beads in the presence of 10 pmol of biotinylated primer was observed with a total binding capacity of 40 pmol of biotinylated oligonucleotide. The binding time was optimized to minimize the time course of the assay using agarose gel electrophoresis to measure the removal of the amplicons from the buffer phase of the binding reaction. Quantitative binding of the biotinylated amplicons was observed within 5 min. of mixing the beads and the DNA samples.

Attachment of the TAP-APC to the amplicon was successfully performed either free in solution before the addition of streptavidin beads or after binding the amplicons to the beads followed by a wash step to remove free primer-αTAP oligonucleotides. The Tₘs for the TAPs range between 55.8-64° C. under the annealing conditions used (150 mM Na⁺). TAP-APC was added at 0.5 μM and incubated at 51° C. for a minimum of 15 min. High stringency was not required to achieve efficient binding. Analysis of the TAPs and α-TAPs indicated that hairpin formation was insignificant under these annealing conditions. The sequence complexity of the reaction was low even in preparations subjected to multiplex PCR. At most 3 pairs of α-TAPs and TAPs are present in a triplex reaction and these sequences can be arbitrarily changed to prevent cross-hybridization. This annealing step was optimized with respect to temperature in a gradient thermocycler and the shortest annealing time was determined using electrophoretic mobility shift of the amplicon as an endpoint.

The PCR reactions were diluted 1:1 in DNA binding buffer to give a final NaCl concentration of 150 mM. The appropriate TAP-APC was added to each DNA sample to a final concentration of 0.5 μM, followed by an incubation at 51° C. for a minimum of 15 min.

Streptavidin magnetic beads were aliquoted to PCR tubes. The beads were washed with 100 μl of 50% (v/v) binding buffer. The washed beads were suspended in the DNA samples followed by incubation at 51° C. for a minimum of 5 min.

The binding reaction was terminated by pelleting the beads with a magnet and removing the binding buffer. The beads were subjected to 2 washes in 180 μl of wash buffer containing the same NaCl concentration as the 50% (v/v) binding buffer (150 mM). Each wash step included a 5 min incubation at 51° C. to replicate the stringency of the binding reaction and then the beads were rapidly pelleted with the magnet. A third wash was in 40 mM HEPES pH 7.5, 40 mM KCl. The beads could be stored refrigerated or could be immediately subjected to Abscription®.

### Step 6: Abscription®

Beads containing bound amplicon:TAP-APCs were pelleted with a magnet to remove storage buffer and were suspended in 10 μl of Abscription® buffer containing 1 mM dinucleotide initiator (GpA), 1 mM NTP (GTP) and 0.4 units of RNA polymerase. One unit catalyzes the incorporation of 1 nmol of NTP in 60 min at 65° C. Abscription® reactions were incubated for 1 hr at 77° C.

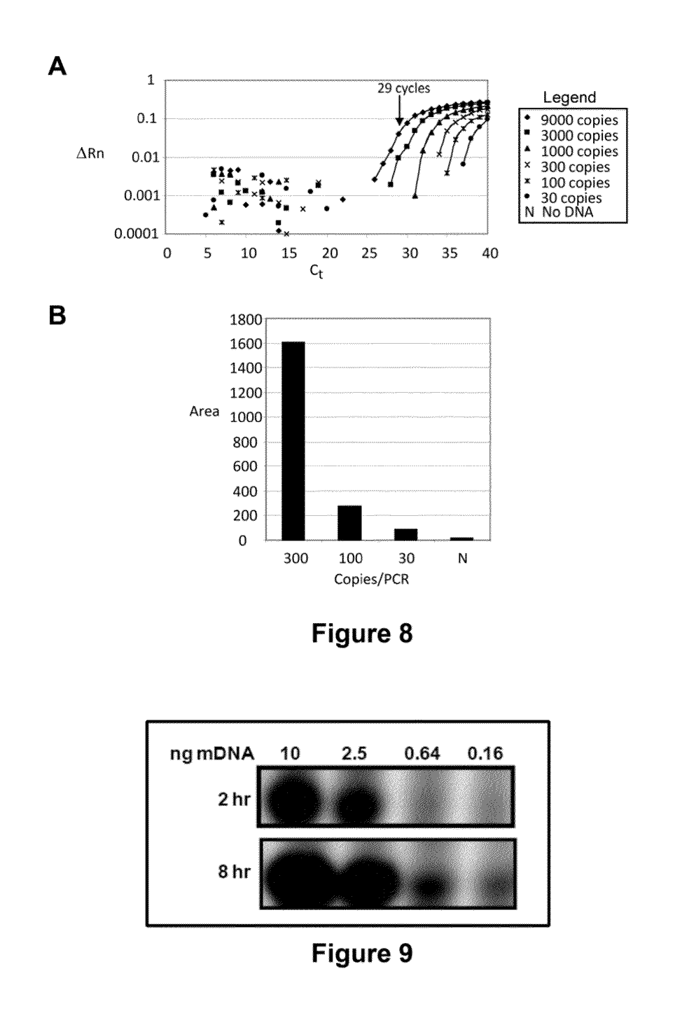

Abscripts were detected by UV-shadowing by spotting 1.5 μl samples onto a silica gel TLC plate containing a fluor. TLCs were developed in an air-tight chamber containing 100 ml of solvent (Isopropanol:Ammonium hydroxide:Activator solution, 6:3:1). Abscripts were detected as dark spots under shortwave UV light as illustrated in FIG. 8B (UV-shadowing).

For LC-MS detection, 10 μl of Abscription® reaction was diluted into 20 μl of HPLC grade water in a 384 well plate. Ten microliters was processed and quantified by LC-MS as illustrated in FIG. 8C.

TABLE 5
Sensitivities of TaqMan ® and Abscription ® assays

| | | Abscription ® (1.5 hr) | | | |
|---|---|---|---|---|---|
| TaqMan ® | | TLC | | LC-MS | |
| Copies | Cₜ | Cycles | LOD (copies) | Cycles | LOD (Copies) |
| 9000 | 28 | 29 | 100 | 29 | 30 |
| 3000 | 30 | | | | |
| 1000 | 32 | | | | |
| 300 | 34 | | | | |
| 100 | 35 | | | | |
| 30 | 37 | | | | |

FIG. 8 shows the results of α-TAP Abscription®-based detection of titrated HeLa compared to Taq® Man PCR using the same priming sites for both methods. 5′-gcgggaccctccagaa-3′ (SEQ ID NO:3) and 5′-actcactggtggcgaagact-3′ (SEQ ID NO:4) were used for the qPCR amplification. 5′-FAM-accacccttataaggctcggaggcc-Iowa Black™ FQ quencher-3′ (SEQ ID NO:5) was the fluorescent probe. 5′-actcactggt ggcgaagact-3′ (SEQ ID NO: 13) and 5′-cctccatcccaaagta[εA]gcgggaccctccagaa-3′ (SEQ ID NO: 12) were the μ-TAP primer pair. FIG. 8B shows the results for TLC detection. Abscription/PCR primers were SEQ ID NO:12 and SEQ ID NO:13. The TAP-APC was made by annealing SEQ ID NO:28 and SEQ ID NO:32. The APC encoded the Abscript GAG. Abscripts were detected using thin layer chromatography (TLC) and UV shadowing. The limit of detection (LOD) at 29 cycles using UV-shadowing was 100 copies. FIG. 8C shows the LC-MS detection results for the same samples analyzed in FIG. 8B. DNA inputs for the PCR reactions varied from 30 to 9000 genomic copies. The LOD for LC-MS at 29 cycles was 30 copies. Area refers to the area of the chromatographic peak containing the Abscript (GAG).

TaqMan® PCR required 35 cycles for detection of 100 copies and 37 cycles for detection of 30 copies (FIG. 8A and Table 5). Abscription® based detection was more sensitive than TagMan® even with TLC and UV-shadowing.

TLC based detection is more sensitive if UV-shadowing is replaced with detection of fluorescent Abscripts. Genomic DNA that was methylated in the CDKN2A CpG island was amplified with primers containing a biotin group (SEQ ID NO:43) and an anti-TAP sequence (SEQ ID NO:42). Amounts of starting genomic DNA were 10 ng, 2.5 ng, 640 pg, and 160 pg. This corresponded to 3000, 750, 188 or 47 copies of genomic DNA. After addition of the TAP-APC encoding AUC, Abscription® was carried out in the presence of the Cy5™ labeled dinucleotide ApU and CTP at 45 C. Samples were withdrawn (1 μA) and analyzed by rapid TLC. Abscripts from 2.5 ng of starting DNA could be visualized after 2 hr of Abscription®, and 47 copies could be detected easily after 8 hours of Abscription® as shown in FIG. 9.

In this experiment, the Abscription® product was the Cy5™-labeled trinucleotide AUC. Cy5™ is actually a rather poor initiator compared to several other fluorescent dyes, reducing the turnover for trinucleotide synthesis to about 8% of that with unlabeled dinucleotides. For this reason, Cy5™ may not be the dye of choice for these assays, but can be replaced with other dyes, such as fluorescein or DyLight (Pierce), both of which give turnovers closer to 35% of that obtained with unlabeled initiators. By using dyes with approximately 4 fold higher efficiency, times may be reduced correspondingly, allowing detection of less than 50 copies of starting DNA in 2 hours. Fluorescein has the additional advantage of being detectable with a low cost, long wavelength UV light.

### Example 6

### Two-Step CpG Island Methylation Detection with APC-Primers

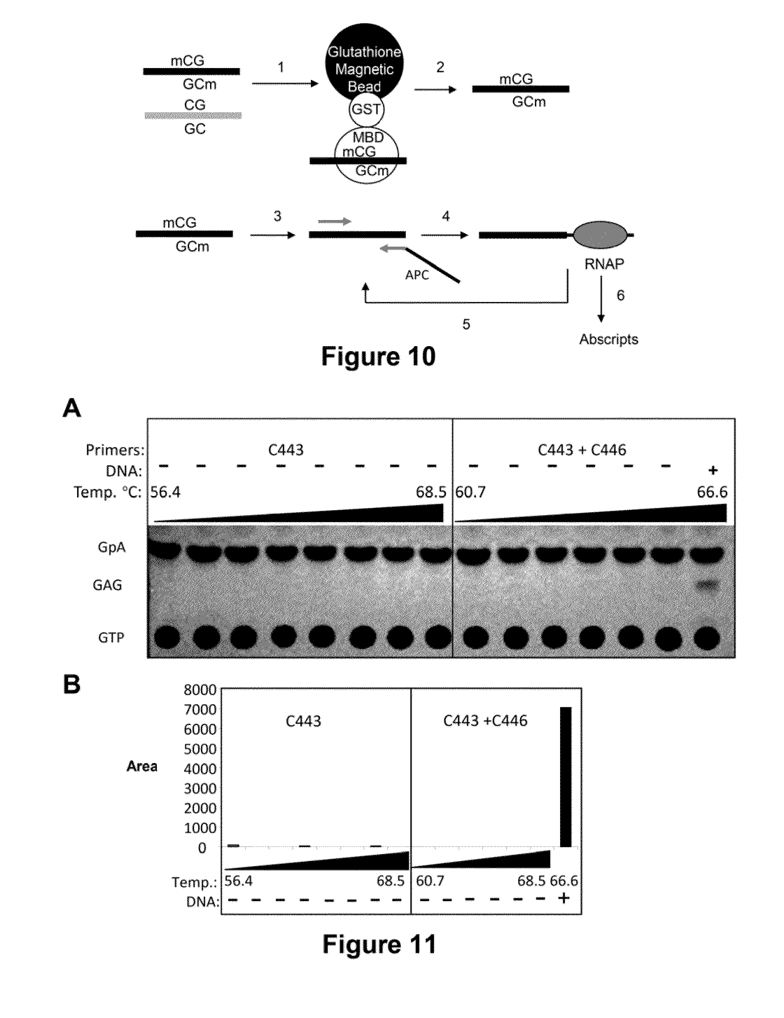

Using a two step detection method, methylated DNAs were isolated from a fragmented genomic DNA sample as in the three-step α-TAP method (EXAMPLE 5). Methylated DNA fragments were bound to immobilized GST-MBD protein (FIG. 10; Step 1) as described above. Methylated fragments were released by exposure to heat or glutathione after washes to remove unmethylated DNA (FIG. 10; Step 2).

Targeted CpG islands were amplified and tagged using a primer that contains an APC sequence at its 5′ end (FIG. 10; Step 3). The single-stranded form of the APC is inactive but becomes activated when it is converted into a double-stranded form during amplification of the target (FIG. 10; Step 4). Thus, Abscription® can be performed during the PCR reaction if initiator(s) NTP(s) and RNAP are included (FIG. 10; Step 6).

### Example 7

### Design and Validation of APC-Primers

APC-primers were designed to avoid self priming and the formation of primer dimers with the reverse primer. At least some of these events are likely to produce active duplex promoters that could would create high levels of background Abscription®. Potential primer sequences were screened as described in EXAMPLE 5 for the potential to form primer dimers and to self prime. The APC portion of the APC primer is 44 nt long of this 33 nt can be changed without significantly affecting Abscription® activity. In most cases potential self priming or primer dimer interactions could be eliminated by changing the sequence of the APC segment of the APC-primer.

Primer pairs that were predicted to be free of potential interactions were tested by performing PCR reactions in the absence of DNA over a range of annealing temperatures to determine if the primers alone could produce background signal. First the APC-primer was tested to determine the level of self priming. Next, PCR reactions without DNA were performed with both the APC primer and the reverse primer to test for primer dimer effects. Completed PCR reactions were supplemented with 1 mM dinucleotide intiator, 1 mM NTP, 0.4 units of RNA polymerase and were analyzed by Abscription®. FIG. 11 shows the Abscription® results for a well designed primer pair (SEQ ID NO:33 and SEQ ID NO:34) that targets the GAPDH CpG island. FIG. 11A shows the TLC data for the APC primer alone (SEQ ID NO:34) and for the primer pair along with a positive control that included HeLa genomic DNA. The encoded Abscript GAG could only be detected in the positive control. FIG. 11B shows LC-MS data for the same samples. Only the positive control produced the Abscript, while the reactions lacking DNA did not produce significant signal over a broad range of annealing temperatures. APC-primer/reverse primer pairs developed for CpG islands are given in Table 6 below.

TABLE 6
APC-Primer/Reverse Primer Pairs

| CpG | | |
|---|---|---|
| Island | APC Primer | Reverse Primer |
| GAPDH | 5′-agagaattttttcataaacattaaatgtacaatgggaa | 5′-ctgcctagggagagaga-3′ |
| (SEQ ID | cgagaaccgtgtgcccaagacc-3′ | SEQ ID NO: 34) |
| NO: 45) | SEQ ID NO: 33 | |
| MGMT | 5′-gctgttgacaattaataaacgctcaatgtacaatggg | 5′-cctgtggtgggcgatgc-3′ |
| (SEQ ID | actgagactcttaggcttctggtggc-3′ | (SEQ ID NO: 36) |
| NO: 46) | (SEQ ID NO: 35) | |
| PTGS2 | 5′-tgcgaaccttgactataaaaattcaatgtacaatggga | 5′-tgtgggaaagctggaatatc-3′ |
| (SEQ ID | cggagaaggtacgaaaaggcggaaaga-3′ | (SEQ ID NO: 38) |
| NO: 48) | (SEQ ID NO: 37) | |
| SNRPN | 5′-gctgttgacacagttcaaacgctcaatgtaaaatggg | 5′-cttgctgttgtgccgttctg-3′ |
| (SEQ ID | acaatcacctccgcctaaaatccctatg-3′ | (SEQ ID NO: 40) |
| NO: 50) | (SEQ ID NO: 39) | |
| GSTP1 | 5′-gctgaagacacagaataaacgatcaatgtataatgg | |
| (SEQ ID | gactgagagcgggaccctccagaa-3′ | 5 -actcactggtggcgaagact-3 |
| NO: 47) | (SEQ ID NO: 41) | (SEQ ID NO: 4) |

### Example 8

### Comparison of Detection of Methylated DNA from Tumor Cell Lines with α-TAP and APC-Primer Methods

PCR reactions were performed with 0.4 units of Hot Start Taq (Fermentas) in the vendor's 1× buffer containing 2 mM MgCl₂, 0.8 mM dNTPs (0.2 mM each), and 5% (v/v) DMSO. One unit of Hot Start Taq incorporates 10 nmol of dNTPs in 30 min at 74° C. The APC-primer and the reverse primer were at 1 μM each. The cycling conditions were 95° C. for 1 min, followed by up to 32 cycles of 95° C. for 30 sec, 62° C. for 30 sec, and 72° C. for 30 sec. A final elongation step was at 72° C. for 5 min. Completed reactions were held at 4° C. Completed PCR reactions (10 μl) were supplemented with 1 mM dinucleotide initiator, 1 mM NTP and 0.4 units of RNA polymerase. Abscription® was then performed for up to 1 hr at 77° C.

Abscription® could be performed during PCR if the dinucleotide initiator and the NTP were included in the PCR reaction at 1 mM each. A thermostable RNA polymerase was added at 0.4 units per 20 μl reaction. One unit catalyzes the incorporation of 1 nmol of NTP in 60 min at 65° C. Abscription® reactions were incubated for 1 hr at 77° C. Abscripts could be detected as described in examples 2 and 5.

TABLE 7
Percent Methylation of Tumor Cell DNAs

| DNA sample | Percent methylation ± SD | | | | | |
|---|---|---|---|---|---|---|
| Detection method | DAPK1 | MGMT | GSTP1 | PTGS2 | GAPDH | SNRPN |
| LNCaP (Prostate) | 81 | 19 | 100 | 60 | 0.8 ± 0.3 | 49 ± 1.5 |
| α-TAP detection | n = 2 | n = 2 | n = 2 | | n = 3 | n = 3 |
| LNCaP | | | 99 ± 1.1 | | 3.3 ± 3.6 | 49 ± 2.9 |
| APC-primer detection | | | n = 4 | | n = 3 | n = 3 |
| MDA-PCA-2b (Prostate) | | | | 66 | 0 | |
| α-TAP detection | | | | | | |
| HeLa (Cervical) | | 96.5 ± 3.0 | | 0 | 4.8 | 48 ± 2.9 |
| APC-primer | | n = 5 | | n = 2 | | n = 7 |

Table 7 shows the results of Abscription®-based detection of methylated DNA from tunor cell lines. Most replicate measurements were done with samples measured once from multiple fractionation experiments. The GAPDH CpG island was unmethylated which was expected if the tumors maintained the normal methylation status in this island. The SNRPN CpG island fit the prediction for an imprinted gene. The other islands were consistent with published results except for DAPK1 in LNCaP for which there are divergent conclusions on its methylation status (Yegnasubramanian et al. (2004) Cancer Res. 64:1975-86; Lin et al. (2001) Am J. Pathol. 159:1815-26; Paz et al. (2003) Cancer Res. 63:1114-21; Toyota et al. (2000) Cancer Res. 60-4044-48; Lodygin et al. (2005) Cancer Res. 65:4218-27).

### Example 9

### CAP Analysis of SNRPN Imprinting

### Materials and Methods

Patient Samples.

De-identified, purified DNAs from peripheral blood samples were obtained from the Molecular Genetics Laboratory, Children's Hospital and Research Center at Oakland under IRB approval. The DNA samples were previously analyzed for the methylation status of the SNRPN imprinting center with MS-PCR (Kubota et al., Nature Genetics Vol. 16, No. 1, pp. 16-17, ISSN 1061-4036, 1997; Kosaki et al., Journal of Human Genetics Vol. 73, No. 3, pp. 308-313, ISSN 0148-7299, 1997).

Separation of Methylated and Unmethylated DNA with a Methyl CpG Binding Domain Protein.

Patient DNAs were fragmented with restriction endonuclease MseI. MseI cuts segments between CpG islands at high frequencies but cuts CpG islands infrequently. Fragmented DNA (7.5-150 ng) samples were fractionated with the MethylMagnet® CpG DNA isolation kit following the instructions in the user manual (RiboMed, MM101K). DNA samples were diluted 5-fold into Binding Buffer and were incubated with 5 μA of GST-MBD magnetic beads for 1 hr at 22° C. with shaking at 1,000 rpm in an Eppendorf Thermomixer. Supernatant fractions were recovered after collecting the beads with a magnet. The beads were washed 2 times in 400 μA of Wash Buffer 2 with 5 min incubations at 22° C. and shaking at 1,000 rpm. A third wash without incubation was performed with 400 μA of 10 mM Tris HCl pH 8, 1 mM EDTA. Methylated DNAs were eluted from GST-MBD magnetic beads by incubation in 80% (v/v) Binding Buffer:18% (v/v) ultrapure water:2% (v/v) NEB buffer #4 at 80° C. for 10 min with shaking at 1,000 rpm in an Eppendorf Thermomixer. The supernatant and the eluted fractions of the binding reactions were saved for CAP analysis.

Coupled Abscription-PCR(CAP) Reaction.

PCR reactions were performed with Maxima Hot Start Taq DNA Polymerase (Fermentas) using the manufacturers reaction buffer [20 mM Tris.HCl pH 8.3 (25° C.), 20 mM KCl, 50 mM (NH₄)₂SO₄] and 2 mM MgCl₂, and 5% (v/v) DMSO. The dNTPS were added to final concentrations of 0. 2 μM each. CAP primers for the SNRPN promoter region were present at 1 μM each. Taq polymerase was added to 2 units/20 μl reaction. Inclusion of DMSO in the PCR reaction was essential to avoid amplification bias against methylated DNA. PCR conditions involved an initial denaturation step at 95° C. for 30 sec, followed by 28 cycles of 95° for 15 sec, 64° C. for 15 sec and 72° C. for 30 sec. A final elongation step was at 72° C. for 5 min followed by indefinite incubation at 4° C. Abscription reactions were set up by supplementing 10 μl PCR reactions with 2 μl of an Abscription master mix consisting of 3.7× Abscription buffer (1× buffer: 40 mM HEPES pH 7.5, 40 mM KCl, 10 MgCl₂), 6 mM dinucleotide initiator [(GpA, GpU or ApU, RiboMed 131, 134, 114], 6 mM NTP (either GTP or CTP) and 1 unit of thermostable Abscriptase (RiboMed MME-1). Abscription was performed in a thermocycler at 77.6° C. for periods ranging from 15 min to 3 hours.

Primer Sequences.

All CAP promoter primers were based on 2 sets of primer sequences listed in Table 8. The SNRPN F1 and SNRPN F2 had 43 nucleotide single-stranded Abortive Promoter Cassettes (APCs) linked to their 5′ ends for the CAP assays. SNRPN F1 and SNRPN R1 were also used for qPCR experiments without the additional APC sequence.

qPCR.

Conditions for TaqMan® qPCR reactions were essentially identical to the PCR portion of the CAP reactions except for the inclusion of ROX reference dye to 300 nM. Primers for the qPCR experiments did not include an APC extension. The primers SNRPN-F2 and SNRPN-R2 targeted the promoter region of SNRPN transcript variant 1. A set of parallel CAP primers were derived from the same primer sequences (Table 8). The probe (5′AGGTATATTGGAGTGATTGTGGCGGG3′ was labeled at the 5′ end with 6-carboxyfluorescene and at the 3′ end with Iowa Black®FQ (IDT). Titrations of HeLa DNA from 10 copies/PCR to 10,000 copies/PCR were amplified in 20 μl volumes in an ABI 7700 Sequence Analyzer.

TABLE 8
Primer sequences for CAP and TaqMan ® assays

| Primer | Assay | Primer Sequence (5′-3′) |
|---|---|---|
| SNRPN F1 | qPCR, CAP | 5′-TGCATAGGGATTTTAG |
| | (APC-primer, | GCGG-3′ (SEQ IN NO:) |
| | Abscript: GAG) | |
| SNRPN R1 | qPCR, CAP | 5′-CCGATCACTTCACGTA |
| | | CCTTC-3′ (SEQ IN NO:) |
| SNRPN F2 | CAP (APC-primer, | 5′-ACCTCCGCCTAAAATC |
| | Abscript: AUC) | CCTATG-3′ (SEQ IN NO:) |
| SNRPN R2 | CAP | 5′-CTTGCTGTTGTGCCGT |
| | | TCTG-3′ (SEQ IN NO:) |

Abscript Analysis: HPLC-Mass Spectrometry (LC-MS).

For high performance liquid chromatography-mass spectrometry (LC-MS) detection of abscripts, 10 μl of the CAP reaction was diluted into 20 μl of HPLC water in a 384 well plate. Volumes of 10 μl were injected into the LC-MS.

Abscript Analysis: Rapid Thin Layer Chromatography (rTLC).

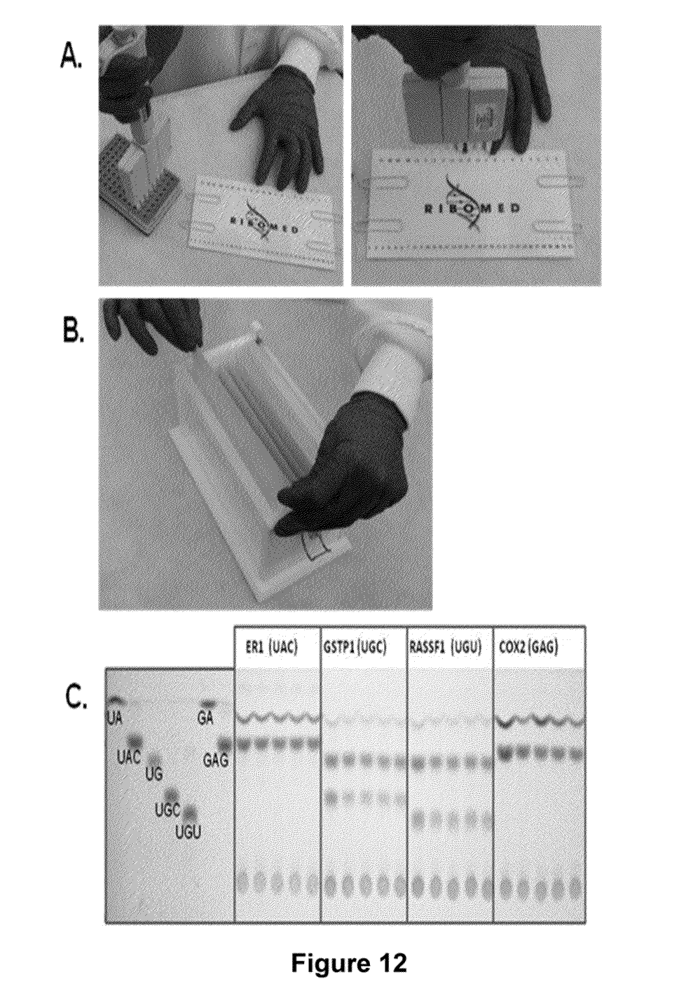

Analysis of abscripts by rTLC is both fast and extremely affordable. All that is needed is a small TLC developing tank and a handheld 254 nm light to visualize the products. Dinucleotides and trinucleotides migrate differently due to their varying polarities. Products are visualized by shining a 254 nm light on the TLC plate, which contains a fluorescent indicator. Nucleotides quench this fluorescence, resulting in a dark spot (UV shadowing). The TLC is photographed with a digital or CCD camera. Alternatively, fluorescent abscripts can be synthesized with labeled dinucleotides and then separated by TLC and visualized with a fluorescent imager or a 336 nm hand-held light (for fluorescein). Analysis by TLC involves three simple steps (FIG. 12).

For rTLC detection of abscripts in the patient samples, 1.5 μA of CAP reaction was spotted 1 cm from the bottom edge of a 10 cm tall silica gel plate containing a UV-excitable fluorophore (Whatman, cat#4420 222). The sample spots were air dried before placing the TLC plate in an air-tight rapid TLC solvent chamber (RiboMed cat#TC6-01) containing 100 ml of freshly made rTLC solvent [6:3:1 (v/v) isopropanol:ammonium hydroxide: Activation buffer 1 (RiboMed, cat AB-1)]. The plate was submerged in solvent at a depth of approximately 5 mm, with the solvent just below the point at which the reaction sample was spotted on the plate. The plate was left in the tank for approximately 20 minutes to allow the solvent to flow upwards through the TLC plate by capillary action, causing separation of the components of the CAP reaction. When the solvent was approximately 1-2 cm from the top of the plate, it was removed from the tank. Developed plates were air dried and photographed with 254 nm UV-illumination.

Step 1. Spot samples (FIG. 12A). Samples (1 to 1.5 μl) are spotted directly onto precut TLC plates. No sample dye is required. The spotting template is designed for use with multi-channel pipettors on one side, allowing 21 samples per plate to be spotted. Even more samples can be analyzed by pipetting individually on the other side of the template, which can hold 32 samples. Step 2: Put plates in the TLC developing tank (FIG. 12B). The TLC chamber holds 6 plates, so 120 samples plus 6 standards can be analyzed simultaneously using the multi-channel pipettor side. The entire process takes less than 1 hour. Up to 192 samples, or the equivalent of two 96 well plates, can be simultaneously processed when using the manual spotting template. Step 3: Irradiate the plate and to visualize the results (FIG. 12C). The TLC takes between 20 minutes to an hour to run, after which the plate is removed, air dried and visualized by UV shadowing, as shown below in (D). Spots can be quantified with the same imaging software used for gels.

Depiction of Prader-Willi and Angelman Syndrome Disorders.

In this EXAMPLE, the methylation status of a DNA segment from +234 to +539 nucleotides downstream from the start-site for SNRPN RNA variant 1 was analyzed to detect Prader-Willi and Angelman Syndrome disorders from blood. The strategy for detecting methylated DNA involved the separation of methylated DNA fragments from the unmethylated versions through the use of the 76 amino acid mouse methyl-CpG binding domain of MBD2 fused to glutathione-S-transferase. The GST-MBD fusion protein, was immobilized to glutathione magnetic beads to allow capture of methylated DNA fragments from a sample as described above. After restriction, the SNRPN RNA variant 1 promoter CpG island was part of a 1,237 nucleotide MseI fragment. There are 62 MseI sites separating this fragment from the next upstream CpG island, two MseI sites separating it from an immediately adjacent cluster of CpG sites which probably are part of the targeted CpG island, and 179 MseI sites before the next downstream CpG island. Experiments with synthetic DNAs showed that MBD2 protein has a strong bias for densely spaced methylated sites. Synthetic DNAs with 4 methylated CpG sites spaced on average 11 nt apart were fractionated with approximately 50% efficiency. Those with six methylated CpG sites were bound quantitatively by GST-MBD2 beads while DNAs having seven to twelve methylated sites spaced between 65 to 91 nucleotides apart were bound very inefficiently (data not shown). Consequently, the linkage of widely spaced methylated CpGs that are not part of a CpG island following MseI digestion is unlikely to significantly influence the fractionation of the CpG island.

### APC-Primer Development

Signal generation in the CAP assay depends on the conversion of the inactive single-stranded APC into an active duplex form. Ideally this is accomplished only when a CAP primer is copied in the course of amplifying the target. The CAP assay is potentially vulnerable to primer dimer effects that would activate the APC in the absence of target DNA. Primer-primer interactions that lead to DNA synthesis copying the APC-primer from the downstream priming segment into the double-stranded and active APC cause strong background signals independent of target DNA. The SNRPN primers were tested for this possibility by performing PCR reactions with, or without DNA over a range of annealing temperatures.

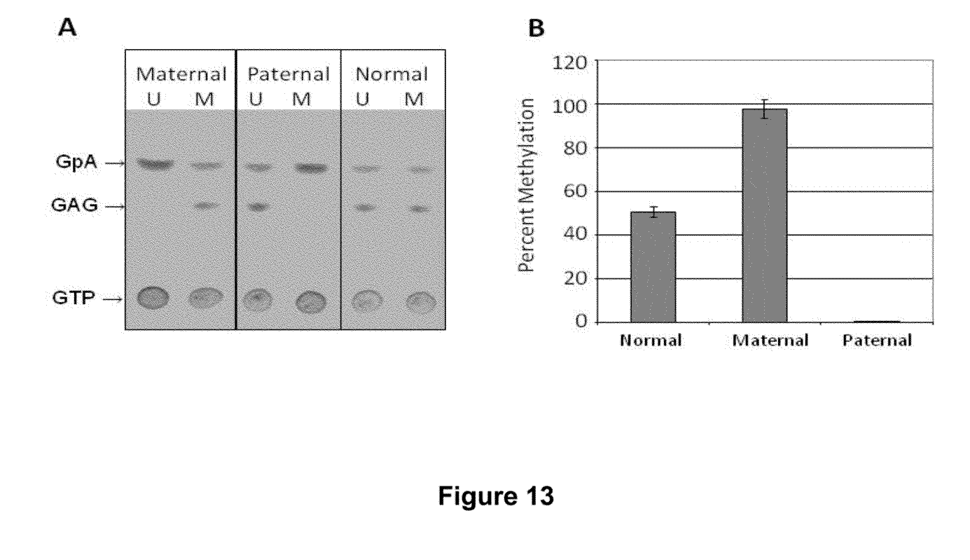

FIG. 13 shows the results for one of the two well-designed SNRPN promoter primer pairs that were used to analyze patient samples (Table 8). PCR reactions were carried out for 30 cycles (2 cycles more than the standard protocol) followed by 30 min of Abscription. No background signal was seen over annealing temperatures from 62.9° C. through 68.5° C. (FIG. 13A). Relatively strong abscript signals were generated in the presence of 1,000 copies of HeLa DNA at annealing temperatures 62.9° C. and 64.9° C. Signal intensity fell at higher stringencies up to 68.5° C. (FIG. 13B).

Primer validation included an assessment of amplification specificity in the presence of genomic DNA. FIG. 13C shows an example for the SNRPN primer pairs that were used to analyze patient samples. PCR reactions were carried out with 3,000 copies of DNA from a normal patient sample and for 32 cycles in order to allow potential non-specific amplicons to be detected by agarose gel electrophoresis. Neither primer pair produced detectable non-specific amplicons.

### Analysis of the SNRPN Imprinting Center in Patient DNAs

The SNRPN assay was applied to the analysis of genomic DNA samples from 38 patients whose diagnosis of PWS and AS were confirmed based on MS-PCR of the SNRPN imprinting center (Kubota et al., supra; Kosaki et al., supra). A collection of 16 normal samples was also analyzed. Purified DNA samples were exhaustively cleaved with MseI in overnight digestions to unlink the targeted SNRPN promoter region from neighboring CpG islands. Treated DNAs were fractionated with GST-MBD magnetic beads without prior purification to remove the restriction enzyme. MseI was inactivated by incubating the samples at 65° C. for 20 min. The supernatants of the binding reactions containing unmethylated fragments were analyzed with the paired fractions containing methylated fragments that were eluted from the beads. The eluted fractions were in the same volume and in the same binding buffer as the supernatant fractions to eliminate potential PCR biases due to sample buffer differences. The fractions were subjected to 28 cycles with 2 alternative sets of CAP primers with APCs that encoded either GAG or GUG. PCR reactions were followed by Abscription reactions for 15 to 30 min. The production of alternative abortive transcripts did not affect the results. The methylation status of a sample was determined by comparing abscript yields for the supernatant fraction (containing exclusively unmethylated SNRPN targets) and the eluted fraction (containing exclusively methylated SNRPN targets).

Both fractions methylated (M) and unmethylated (U) patient samples were amplified for the SNRPN imprinting center with CAP promoter primers and then were subjected to Abscription to produce the abscript GAG. FIG. 13A shows representative rTLC results for samples showing exclusively methylated SNRPN DNA (PWS), exclusively unmethylated SNRPN (AS) or an equal representation of both methylated and unmethylated DNA (Normal). GTP remains at the origin during the development of the chromatogram while GpA and GAG are separated from each other based on their differing rates of migration in the rTLC solvent. FIG. 13B shows a quantitative summary of all the CAP assays with LC-MS detection of the abscript signals. Normal samples (n=16) showed 50.4%±2.5 methylation, PWS (Maternal pattern) samples (n=25) showed 97.5%±4.2 methylation and AS samples (n=13) showed 0.23%±0.31 methylation. Methylated DNA inputs were between 50 ng to 150 ng of DNA.

Quantitative detection of methylated DNA was performed by LC-MS of diluted Abscription reactions. CAP reactions were fractionated by HPLC to separate the dinucleotide initiator from the trinucleotide abscript. The outflow from the HPLC column was injected into an electrospray ionization mass spectrometer (Waters LCT-Premier) to generate a chromatographic profile based on the mass/charge ratio of the abscript. The area of the abscript chromatographic peak is linearly related to the amount of abscript. FIG. 13B and Table 9 show summary data for all the samples grouped into Normal, Maternal (methylated) and Paternal (unmethylated) patterns. The percent methylation was based on the abscript amount in the eluted fraction divided by the total abscript amount (the sum of the supernatant and eluted fractions). The results were highly reproducible despite the fact that the averages were based on multiple individuals. The large variance for the methylation level of the Paternal pattern was probably due to the statistics of sampling because the level of methylation indicates that the signals were probably generated from fewer than 5 molecules per CAP reaction based on estimates of the input amounts in calibrated CAP assays.

The analysis of patient samples also indicated that nonspecific amplification was insignificant. A non-specific amplicon that is not linked to the SNRPN target would skew the results of the normal DNA samples away from a 1:1 ratio of methylated to unmethylated target DNA. If a hypothetical secondary target was methylated the paternal methylation pattern would show a proportionately high background of methylated signal. The opposite bias would be seen in the maternal methylation pattern if the secondary target was unmethylated. As shown below, all of the patient samples fit one of the PWS, AS, or Normal methylation patterns without significant backgrounds (Table 9). The primer sequences that were the basis for the APC-primer pair SNRPN F2, R2 assay showed no apparent bias for unmethylated DNA over methylated DNA. For example the average amplification efficiencies over a 10° C. annealing temperature range were 92.7%±7.0 (n=11) for HeLa DNA and 92.9%±5.5 (n=4) for artificially methylated HeLa in TaqMan® assays.

TABLE 9
Cumulative results for Prader-Willi/Angelman Syndrome Samples

| Methylation Pattern | Percent Methylation ± SD | No. samples | 95% confidence interval |
|---|---|---|---|
| Normal | 50.4 ± 2.5 | 16 | 1.23 |
| Maternal | 97.5 ± 4.2 | 25 | 1.65 |
| Paternal | 0.23 ± 0.31 | 13 | 0.17 |

The reproducibility of the fractionation method was evaluated by performing between 4 to 7 independent fractionations on DNA from 4 normal samples (Table 10). All of the fractionation runs gave good reproducibility with coefficients of variation (CVs) between 1.2% and 6.3%. The use of the alternative primers did not have a significant quantitative effect on the detection results. Sample #41 was analyzed in triplicate with APC-SNRPN F2, R2 that encoded AUC instead of GAG. The average methylation level was 49.8%±0.69 SD in agreement with the result for sample #41 in Table 10. The single-blinded assignments of individuals to the 3 groups based on TLC and LC-MS were 100% concordant with earlier classification of the samples based on MS-PCR (data not shown).

TABLE 10
Reproducibility of Fractionation ¹Data from Table 9

| | ¹Cumulative | Individual Normal Samples | | | |
|---|---|---|---|---|---|
| | Normal | #36 | #39 | #41 | #53 |
| Percent Methylation | 50.4 | 50.5 | 47.0 | 49.0 | 46.3 |
| SD | 2.5 | 0.58 | 2.6 | 1.02 | 2.9 |
| CV | 4.9% | 1.2% | 5.6% | 2.1% | 6.3% |
| No. Fractionations | 16 | 4 | 7 | 7 | 4 |

### Sensitivity of CAP Verses TaqMan

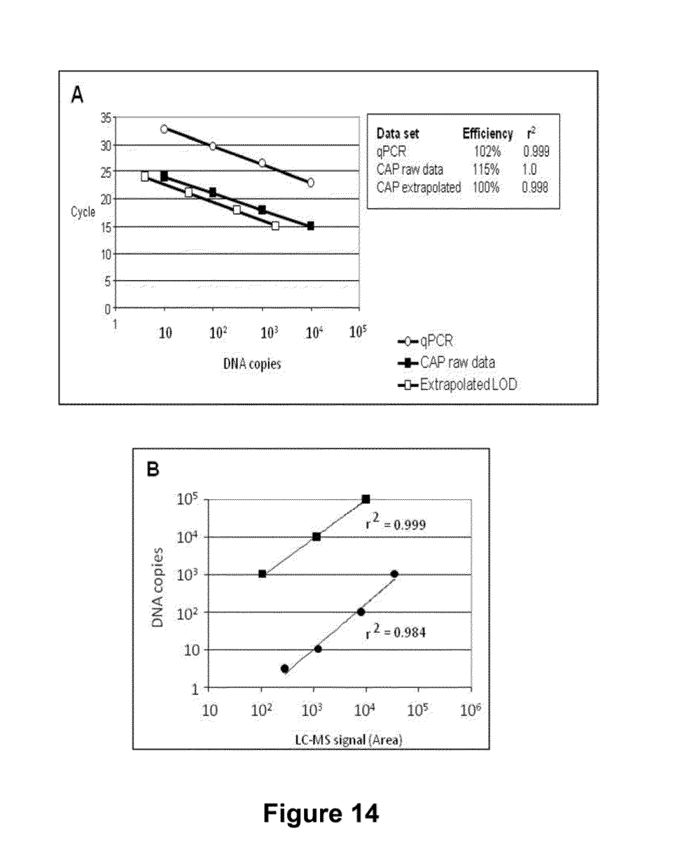

Coupled Abscription-PCR involves the addition of Abscription, a linear signal amplification step, to PCR and is therefore much more sensitive than PCR alone. The relative sensitivity of the CAP assay was compared to a TaqMan PCR assay using the same SNRPN priming sites and PCR cycling conditions (FIG. 14). FIG. 14A shows a plot of Ct verses DNA amount for TaqMan amplification of HeLa DNA along with analogous plots for CAP. In the case of the CAP assay, the Ct-analog for the raw data is interpreted as the minimal cycle number required for detection of a particular input DNA copy number. These DNA amounts were not equivalent to limits of detection because the signals overshot the threshold for detection (an LC-MS signal of 100) by amounts ranging up to 549. The actual limits of detection at various cycles were calculated by extrapolating the lines relating LC-MS signals and DNA copy numbers to the detection threshold of 100 for each endpoint experiment. These extrapolated DNA copy numbers were plotted with the associated end point cycles in FIG. 14A (unfilled squares).

The sensitivity of the CAP assay can be adjusted with variations in the Abscription time following the PCR step. Overall a 3 hr Abscription reaction resulted in detection with a reduction of 11 cycles compared to the TaqMan assay. This translates to about a 2.000-fold improvement in sensitivity assuming 99.7% amplification efficiency. Sensitivity can also be estimated by determining the number of target copies at the detection threshold. The number of amplicons, Nt, at the Ct can be calculated by extrapolating the number of cycles to 0 in FIG. 14A, where the x intercept is interpreted as the number of targets at the detection threshold (Rutledge & Cote, 2003). The qPCR assay produced an Nt of 6×10¹¹ while the normalized CAP Nt was 4×10⁷, a 2.650-fold improvement in sensitivity with a 3 hr Abscription period. A 15 min Abscription time produced a 64-fold improvement in sensitivity over qPCR (FIG. 14A).

The dynamic range of the CAP assay could be made to extend over approximately 5 orders of magnitude by performing two CAP reactions at high and low cycle numbers as shown in FIG. 14B. Samples with 10,000 and 100,000 DNA copies are prone to underestimates of DNA amount at high cycle numbers due to depletion of the Abscription reagents at moderate Abscription reaction times. The high end of the dynamic range is accurately quantified by performing 20 PCR cycles coupled with 15 min of Abscription. At the low end of the range (DNA input <1,000 copies), PCR is performed for 28 cycles followed by an Abscription time between 15 to 30 min. Following this strategy we were able to consistently quantify DNA amounts between 3 molecules to 100,000 molecules.

### Analysis of DNA Methylation in Other Bodily Fluids

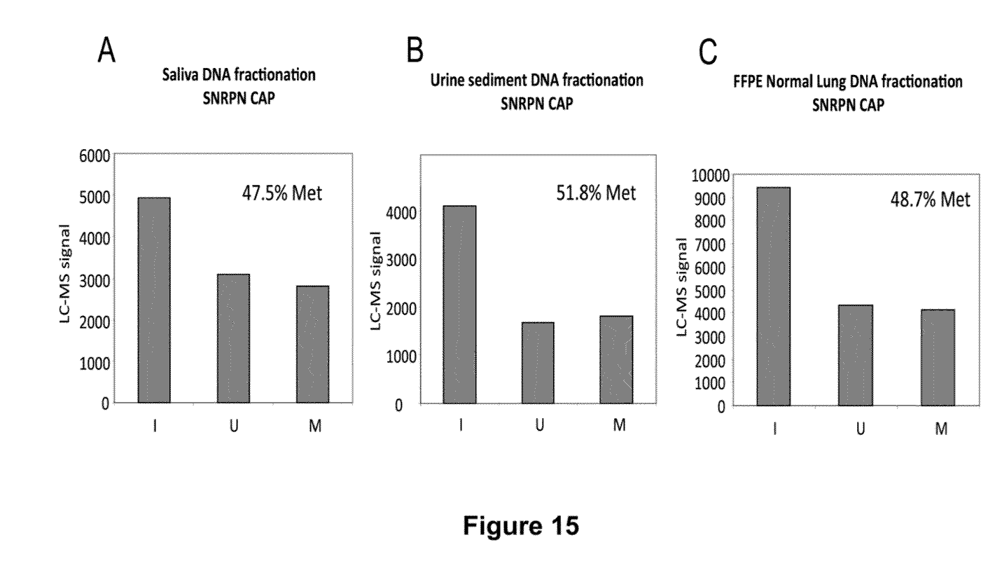

The methods of the present invention are extremely robust, sensitive, rapid and quantitative method to analyze even small changes in DNA methylation levels of differentially methylated regions. It works well and reproducibly in on DNA isolated from saliva and urine (FIGS. 15 A and B), in which the normal methylation pattern is 50% methylated and 50% unmethylated. In addition, the process works well and reproducibly on DNA from Formalin-Fixed, Paraffin-Embedded (FFPE) tissue (FIG. 15C), where bisulfite based methods often fail.

### Comparison of CAP Results to Bisulfite Sequencing

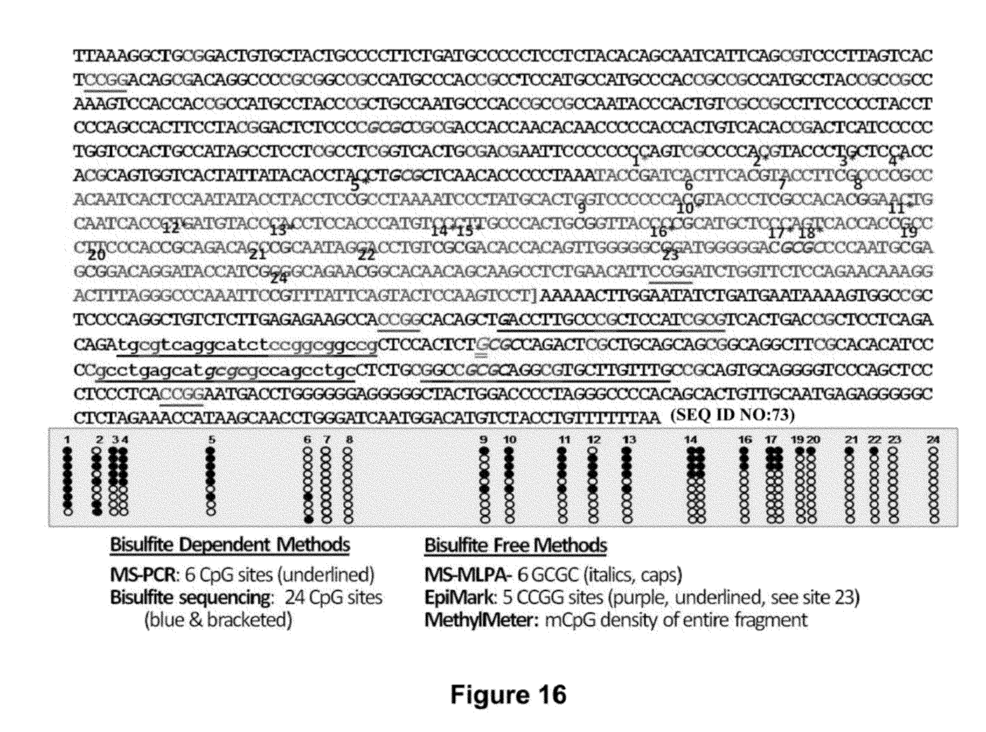

Although the results obtained by the CAP methods of the present invention were 100% concordant with the results from MS-PCR, the method was further validated by analyzing SNRPN methylation in HeLa DNA (50%) and comparing to bisulfite sequencing. The region of SNRPN CpG island that was analyzed with bisulfite sequencing is shown in FIG. 16.

The entire island that was probed is shown in FIG. 16. The region that was sequenced is in the middle of the island and the CpG sites that were interrogated are numbered 1 to 24. The results of the bisulfite sequencing are shown under the sequence. A black circle indicates a methylated site. Sites that are used in MS-PCR analysis and some restriction enzyme based methods are shown. Note that site number 24, which was determined to be unmethylated by pyrosequencing, is the site used in the commercially available method EpiMark™ (NEN), which uses a restriction enzyme that cleaves differentially at this site based on CpG methylation, so this method would have incorrectly scored all of these samples as unmethylated.

Because Methyl Binding Domain (MBD) based fractionation does not damage DNA, less sample is required than is needed for methods that rely on bisulfite treatment. MBD based DNA fractionation has been successfully used with as little as 1 ng of genomic DNA. DNA amounts in the microgram range can be processed by scaling-up the volume of MBD-magnetic beads in the binding reaction.

The linkage of a linear signal amplification to the target amplification of PCR in the CAP method greatly increases assay sensitivity without adding complexity to assay development. Modest Abscription times between 15 min to 30 min allowed a reduction of between 6-7 cycles compared to TaqMan® assays on undamaged DNA. The CAP approach shows a greater relative advantage in sensitivity compared to the application of TaqMan® assays to bisulfite treated DNA. Primer development for CAP assays is free of the constraints associated with bisulfite assays. CAP primers do not have to overlap with CpG sites nor do they have any constraints on primer spacing since no intervening probe sequence is needed. Although care must be taken to avoid primer-dimer effects that lead to activation of the APC in the absence of DNA, potential problems can be identified with conventional primer development software that shows homodimer- and heterodimer interactions. The potential for priming events can be eliminated by changing nonconserved promoter sequences or by choosing a new reverse primer. Abscription provides an extremely sensitive assay to confirm the absence of primer-dimer effects and to identify optimal stringency conditions.

There are several options for abscript detection that depend on the nature of the targets. In the case of imprinting disorders where classification of samples is based on a simple yes or no result, a qualitative approach can be taken with rTLC. This method has the advantage that multiple samples can be processed rapidly in parallel allowing for greater through-put than LC-MS where samples are processed sequentially. All of the PWS AS samples gave unambiguous results that allowed them to be correctly classified based on visual inspection (FIG. 13A). LC-MS is better suited for analysis of methylation in tumor cell DNA where quantitation is important or where it is important to monitor changes in methylation levels over time.

### Example 10

### CAP Assay for Glioma-CpG Island Methylator Phenotype

Gliomas are divided by the World Health Organization into grades based on histopathology. Recent molecular analysis of CpG methylation showed that a Glioma-CpG Island Methylator Phenotype (G-CIMP) is associated with the methylation of hundreds of genes. A stable G-CIMP profile involving 8 genes is linked to enhanced survival in grades 2-4. Although bisulfite-based methods were used to develop and validate the 8-gene G-CIMP pattern, bisulfite-based methods have a low frequency of technical success in a clinical laboratory setting where limited amounts of Formalin-Fixed, Paraffin-Embedded (FFPE) tissues are available. The high failure rate with FFPE DNA is due to the addition of bisulfite-induced DNA damage to the extensive DNA damage caused by formalin fixation. The present invention provides a bisulfite-free G-CIMP assay that relies on the separation of methylated DNA from unmethylated DNA based on a methyl-CpG binding protein immobilized to magnetic beads. The bead-bound fraction and the unbound unmethylated fraction are amplified with a primer pair that includes an inactive promoter extension. Amplification of a target converts the promoter into an active duplex which can generate signals in the form of highly defined abortive RNAs. The methyl-CpG binding protein assay-based assay of the invention replicated the G-CIMP+ assignments on samples that were used in the original G-CIMP+ validation using bisulfite-based methods.

### Materials and Methods

Glioma Samples.

De-identified FFPE samples on glass slides were obtained from the Department of Pathology, The University of Texas, MD Anderson Cancer Center, Houston, Tex.

DNA Purification from FFPE Tissue.

FFPE tissues scraped from glass slides and were deparaffinized in 1.7 ml microcentrifuge tubes by 2 incubations with 1 ml of xylene at 50° C. for 5 min each. All solution changes were made by centrifugation at 14,000×g for 5 min at room temperature. Residual xylene was removed by 2 washes with 1 ml of 100% ethanol. Following the removal of the ethanol, the tissues were dried at 37° C. for 15 min. The tissues were suspended in 1 ml of 1M NaSCN followed by a 3 hour incubation at 37° C. The NaSCN was removed by centrifugation after the addition of 0.5 ml of ultrapure water to reduce the density of the solution. The tissues were washed 3 times with 1 ml of wash buffer (50 mM Tris-HCl pH 8.5 at 25° C., 100 mM NaCl, 1 mM EDTA, 0.5% (v/v) Tween 20 and 0.5% (v/v) Nonidet P-40). The tissues were then digested at 55° C. for 3 hours in 0.2 ml of wash buffer supplemented with 20 mM DTT and 100 μg of Proteinase K. An additional 100 μg of Proteinase K was added and the incubation was continued for 30 min at 55° C. Proteinase K was inactivated by incubation at 80° C. for 20 min followed by ethanol precipitation at −20° C. for 2 hours with 0.5 vol of ammonium acetate and 2 vol of 100% ethanol. The DNA pellets were recovered by centrifugation and were washed with 1 ml of 70% ethanol. The dried pellets were dissolved in 50 μl of TE buffer (10 mM Tris-HCl pH 8 at 25° C., 1 mM EDTA).

AluI Digestion and Validation.

Purified DNA was digested with AluI (10 units, New England Biolabs, Beverly Mass.) in an optimized 1× reaction buffer (20 mM Tris-HCl pH 8 at 25° C., 50 mM KCl, 5 mM MgCl₂, 1 mM DTT, RiboMed Biotechnologies, Carlsbad, Calif.) that was compatible with the downstream Methyl CpG Binding Protein DNA fractionation step. Digestions were performed for a minimum of 6 hours at 37° C. AluI was inactivated by incubation at 65° C. for 20 min.

The extent of AluI cleavage was evaluated by amplifying a sample of the AluI digest and an uncut control from the same reaction mixture with a primer pair that is unable to amplify AluI cut human DNA (5′-promoter-AGGCACTCCCTCACGGGGTC, 5′-GAGGGCTGCGGGCGAACTAG). The relative amounts of amplicons from the cut and uncut samples were determined by abscription as described below. The fraction of AluI cut DNA was expressed as 1-(cut DNA signal/uncut DNA signal). The concentration of amplifiable DNA was determined with the use of a titration of uncut HeLa DNA that was amplified in parallel with the samples.

DNA Fractionation.

Methylated DNA was purified with a modified version of the MethylMagnet® mCpG DNA Isolation kit (RiboMed Biotechnologies, Carlsbad, Calif.). Samples containing 10 ng of amplifiable DNA were combined with equal volumes of 2× Binding Buffer and then were mixed with 5 μl or 10 μl aliquots of magnetic beads containing a Glutathione-S-Transferase-Methyl-CpG-DNA-Binding-Domain fusion protein (GST-MBD). The 10 μl bead inputs were used with DNA inputs greater than 500 ng. The beads were washed with Wash Buffer 1 before adding them to the DNA. The bead-DNA mixtures were incubated at RT for 1 hr with mixing sufficient to keep the beads suspended. The supernatant of the binding reaction containing unmethylated DNA was recovered after the beads were immobilized with a magnet. The beads were then washed 2 times in 0.4 ml of Wash Buffer 2 with mixing for 5 min. After a final static bead wash with 0.4 ml of TE buffer without incubation, the beads were suspended in a volume of Bead Fraction Buffer equivalent to the original DNA input volume. Bead Fraction buffer was identical to the input buffer which contained the unmethylated supernatant of the binding reaction.

PCR.

Both the MethylMagnet supernatant (unmethylated DNA) fraction and the bead bound fraction (methylated DNA) were subjected to PCR in 8 separate reactions each containing a different promoter primer pair associated the G-CIMP marker panel (Table 11). Reactions were carried out in 20 μl volumes containing 1×PCR buffer (20 mM Tris-HCl pH 8.3 at 25° C., 20 mM KCl, 5 mM (NH₄)₂SO₄) supplemented with 1.5 mM MgCl₂, 0.25 μM to 0.5 μM of each primer, 0.8 mM dNTPs, 6% (v/v) DMSO, 15 μM CpU (an internal standard for abscription) and 1 unit of chemically modified Hot Start Taq (Maxima Taq, Fermentas, Glen Burnie, MD). Supernatant fractions (2 μl) and suspended Bead fractions (2.06 μl) were added to 18 μl of master mix. The increased volume of the Bead fraction compensated for the volume occupied by the resuspended beads.

The cycling conditions were: step 1, 94° C. for 2 min; step 2, X° for 30 sec; step 3, 72° C. for 30 sec; step 4, repeat steps 1-3 two times; step 5, 94° C. for 30 sec; step 6, X° for 30 sec; step 7, 72° C. for 30 sec; step 8. repeat steps 5 to 7 from 25 to 31 times depending on the primer pair; step 9, 72° C. for 5 min; step 10; 4° C. hold. Depending on the specific primer pair, the annealing temperature X° was 58° C., 61° C. or 63° C.

Table 11 lists the priming segments of the promoter primers and the complete sequences of the reverse primers for all of the G-CIMP targets.

TABLE 11
G-CIMP Primer Sequences

| | Forward primer | |
|---|---|---|
| Target | (Promoter primer)* | Reverse primer |
| ANKRD43 | GCCCGCAGGGACCGCTTCAA | CCTTCACCACCGCCACGTTGT |
| DOCK5 | TGTAGCAGCCTTAGTCGCCG | GCGCACTCACCAACCCCGTA |
| | CC | |
| FAS | ATTGATTCAGCAACTTGGCC | TTTGTGCAACGAACCCTGAC |
| | TGCG | TCCT |
| HFE | CGGCGCTTCTCCTCCTGATGC | CAGCCCTCGGACTCACGCAG |
| LGALS3 | TTTGATTATCGAGGGCGCTG | ACGTGTGTGGGTCTCGTAAG |
| | GCGTT | GT |
| MAL | GGATCCCAGCGCCGAACCAG | CTTCCGCGTCCACTGAGCCG |
| RHOF | ACTGAGGCTGGGAGGTCGGC | CGGGCACTAGCGGAGCCAAG |
| | | GGGGCATCCATTGCCCGGAG |
| WWTR1 | GGAAGCCCGAGGAGCCTGGA | TCTCGGGACTCACCCGAGCG |

*Only the priming segment of the promoter primer is shown

Abscription.

An Abscription master mix was composed of 148 mM HEPES pH 7.4, 148 mM KCl, 37 mM MgCl₂, 6 mM NpN, 6 mM NTP and 0.06 vol of Abscriptase (RiboMed Biotechnologies, Carlsbad, Calif.). The dinucleotide initiator NpN was GpA, GpG, GpU or UpU depending on the promoter sequence attached to the amplicon. GpG was paired with UTP while GpA, GpU and UpU were paired with GTP. The Abscription master mix (2 μl) was combined with 10 μl of PCR reaction. Abscription was performed for 30 min at 76.4° C.

LC-MS Detection.

Abscription reactions (10 μl) were diluted with 20 μl of HPLC grade water in a 384 well plate. Sample volumes of 10 μl were injected into an LCT-Premier LC-MS (Waters Corporation, Milford, Mass.). Chromatographic peaks corresponding to abscript m/z values and known retention times were integrated to quantify the amounts of abscripts. The CpU internal standard was used to correct for pipetting and sampling variances in the post-PCR steps of the assay. The abscript signal was multiplied by the ratio of the average CpU signal divided by the sample CpU signal. The percent methylation was based on the ratio of the background-corrected Bead fraction signal to the sum of the Bead fraction and Supernatant fraction signals.

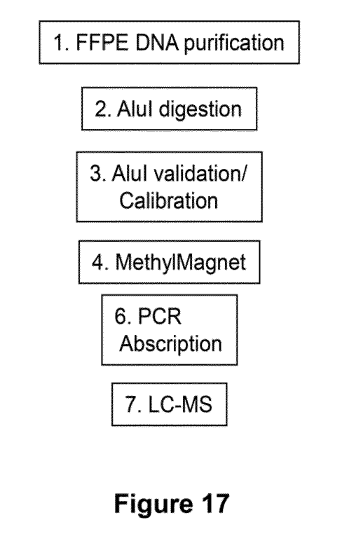

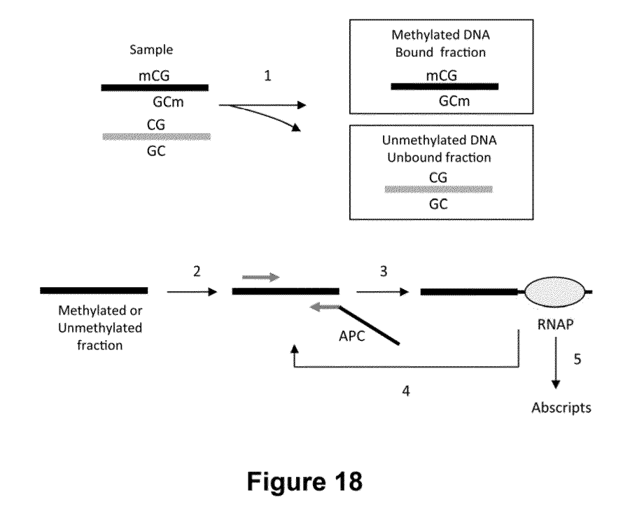

FIG. 17 shows the work flow for the MethylMeter® assay, DNA methylation detection assay of the invention. The method relies on the separation of methylated DNA from unmethylated fragments with the use of magnetic beads bearing a GST-MBD fusion protein. The presence of targeted genomic segments in the methylated and unmethylated DNA fractions is detected by target amplification by PCR followed by linear signal amplification through abscription. Signal amplification is made possible by incorporating a promoter sequence at the 5′ end of one of the primers. The inactive single-stranded promoter is converted into an active duplex form in the course of amplifying the target. Linear signal amplification is accomplished with the addition of the Abscriptase enzyme which performs abortive transcription to produce a trinucleotide abortive transcript or Abscript (FIG. 18). Abortive transcription is limited to the production of a specific trinucleotide by the inclusion of a dinucleotide initiator and a single ribonucleoside-triphosphate. Abscripts can be detected by thin-layer-chromatography, capillary electrophoresis or mass spectrometry.

A collection of highly abortive promoters called Abortive Promoter Cascettes (APCs) have been developed that collectively produce 20 trinucleotide abscript signals. Trinucleotide synthesis by abortive transcription is highly efficient because only a single synthesis step is required and the Abscriptase does not dissociate from the template between rounds of synthesis.

DNA Preparation.

FFPE patient DNA was purified from glass slides followed by digestion with AluI. Sonication or cleavage with restriction endonucleases is normally performed prior to fractionation of undamaged DNA to avoid biases in the DNA binding reaction caused by untargeted methylated CpGs in the vicinity of a CpG island. We attempted to maximize concordance between the assay results and the bisulfite-based real time PCR(RT-PCR) method that was used to establish the G-CIMP profile (Noushmehr et al, Cancer Cell. 17(5):510-22, 2010).

Because the original RT-PCR method interrogated a limited number of clustered CpG sites, we chose to fragment the CpG islands to generate small target fragments that either were adjacent to, or included these CpG sites (Table 12). In spite of the fact that FFPE DNA is highly nicked, the target islands were fragmented with AluI. Due to the staggered distribution of nicks, even heavily damaged DNA preparations have a significant fraction of duplex segments that are at least as large as a complete CpG island. Highly nicked, long duplex molecules are expected to be fractionated by the GST-MBD beads as efficiently as intact DNAs of identical length. The AluI cleavage step was performed to protect against potential biases caused by neighboring methylated CpGs in a heterogeneously methylated island. Heterogeneous methylation could cause CIMP⁻ islands to be assigned CIMP⁺ status if the targeted region is unmethylated but a distal portion of the island is methylated. We encountered this effect for several CpG islands when AluI cut and uncut DNAs of CIMP⁻ sample 12-039 were compared (Table 13).

TABLE 12
Targeted CpG island AluI fragments

| Target | # CpGs | AluI fragment size | Distance from TSS* |
|---|---|---|---|
| ANKRD43 | 15 | 108 | 0^{†} |
| DOCK5 | 27 | 212 | +89 |
| FAS | 13 | 302 | 0 |
| HFE | 15 | 144 | +150 |
| LGALS3 | 63 | 555 | +87 |
| MAL | 18 | 193 | −456 |
| RHOF | 24 | 247 | 0 |
| WWTR1 | 31 | 564 | +45,523 |

*Upstream (−), or downstream (+) distance of the AluI fragment from the Transcription Start Site (TSS)
^{†}A distance of 0 indicates that the TSS is within the AluI fragment

TABLE 13
Effect of AluI cleavage on methylation calls for CIMP⁻ sample 12-039

| Physical State | Percent Methylation | CpG Island |
|---|---|---|
| AluI cut | 8.8 | ANKRD43 |
| Uncut | 47 | ANKRD43 |
| AluI cut | 17 | HFE |
| Uncut | 40 | HFE |
| AluI cut | 31 | LGALS3 |
| Uncut | 53 | LGALS3 |
| AluI cut | 20 | MAL |
| Uncut | 45 | MAL |

Digestion with AluI produced fragments for the 8 targeted genes between 564 and 108 nt in length. The number of CpG sites sampled by the invention assay within these fragments ranged from 12 to 63 (Table 12).

Primer Design.

Primer design for the methylation assay of the invention was subject only to limitations on the template size due to the short single-stranded template lengths in heavily damaged FFPE DNA, and the need to avoid AluI sites. Because the abscription based assay does not have to accommodate additional probes, the primers could be spaced at arbitrarily short distances. Template lengths for the 8 G-CIMP primer pairs varied from 43 to 97 nt.

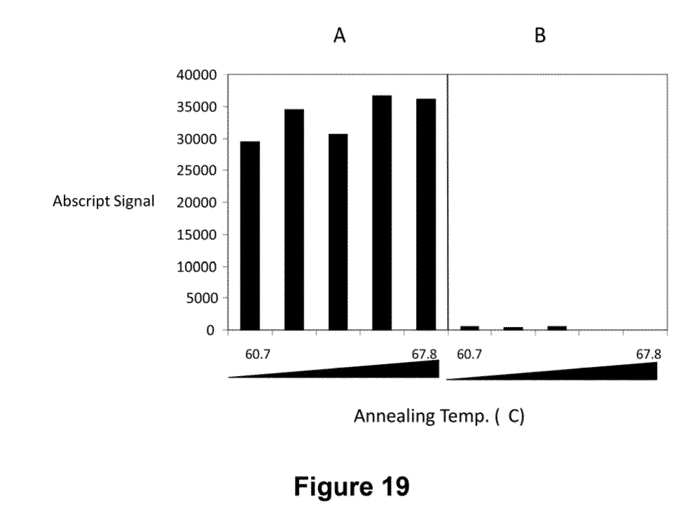

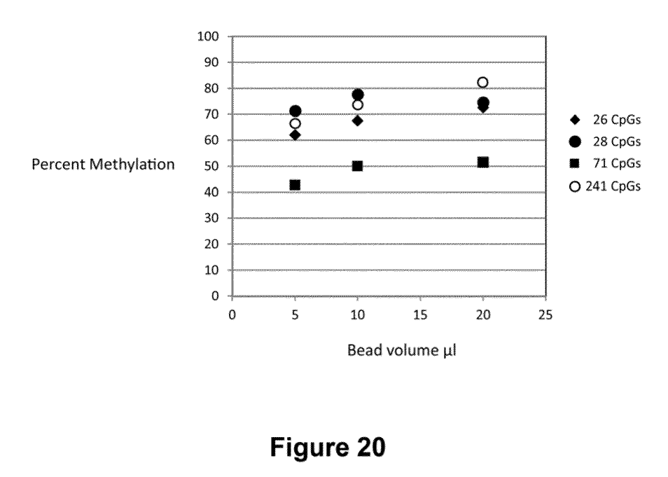

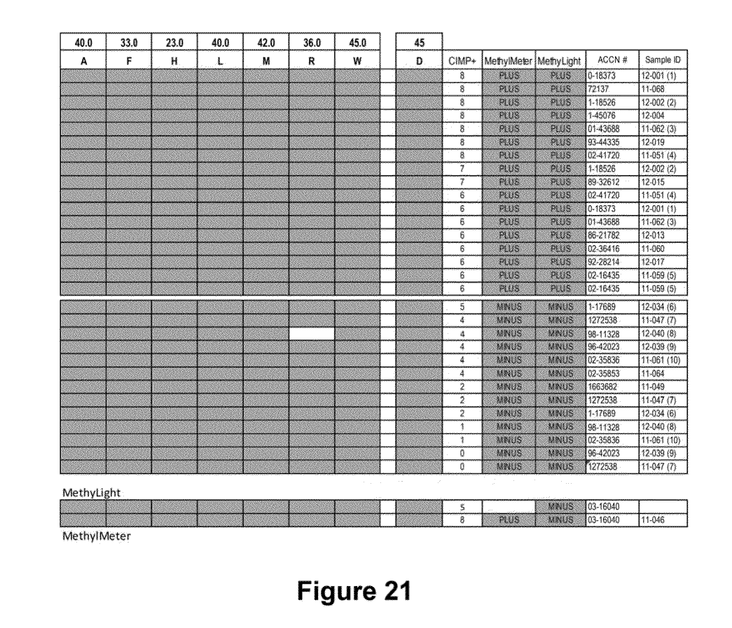

The method of the present invention is potentially susceptible to high background signal due to primer-primer interactions that might lead to activation of the APC. Potential primer-primer interactions were screened in silico followed by sequence changes to the APC segment to eliminate potential primer interactions. Candidate primer pairs from the in silico screen were screened further by PCR-abscription over a gradient of annealing temperatures in the presence of HeLa DNA (150 genomic copies/PCR) and in the absence of DNA. The promoter-primer system was a sensitive means to identify priming sites that were associated with minimal primer-primer interactions. FIG. 19 shows an example of the annealing gradient screen of the HFE primer pair.

Sensitivity.

We previously showed that PCR linked to abscription is up to 2000 fold more sensitive than TaqMan® PCR (ref McCarthy et al., “MethylMeter™: A quantitative, sensitive, and bisulfite-free method for analysis of DNA methylation”, in DNA Methylation, Tatarinova ed, ISBN 979-953-307-453-4 (In Press)). Combining this advantage with the absence of bisulfite-induced damage, the methods of the present invention allow successful processing of FFPE samples that cannot be analyzed by bisulfite-dependent PCR methods.

The sensitivity limit for the invention assay was largely set by the statistics of sampling when setting up PCR reactions for the methylated and unmethylated fractions. An input of 2 ng of amplifiable DNA into a total of 40 μl of binding buffer allows a maximum of input of 30 DNA copies in the PCR reaction for either fraction. Successful amplifications for all 8 target genes was performed with the sample having the smallest amount of amplifiable DNA (4 ng of amplifiable DNA, FIG. 19, sample 11-062, CIMP⁺).

Because the FFPE DNA is heavily damaged only a small percentage is amplifiable. We observed an amplifiable fraction of between 0.6% to 10.3% among our FFPE DNA preparations. In order to reach an input standard of 10 ng of amplifiable DNA into the overall procedure, up to 630 ng of total DNA input was required. This level of total DNA did not exceed the binding capacity of the GST-MBD beads at bead volumes used in the validation as shown in FIG. 19. At an extreme total DNA input of 780 ng, the estimates of methylation percentages using 5 μl of GST-MBD beads were between 80% to 95% of the estimates using 25 μl of beads. Fragments containing as few as 26 CpGs competed for binding with the total methylated DNA input as effectively as fragments containing 241 CpGs.

Detection of the 8-Gene CIMP Pattern in Clinical FFPE Samples.

A total of 20 FFPE glioma samples of grades 2-4 were processed with the invention assay as described above. These samples were previously analyzed for methylation pattern at the MD Anderson Cancer Center using MethyLight, a bisulfite dependent real time MS-PCR method (Noushmehr et al, supra). We used the same CIMP markers that were used in the original validation except that Noushmehr et al., supra) tested FAS at 2 closely spaced sites while we tested FAS with a single fragment that included all of the previously tested CpGs. The CIMP⁺ pattern was assigned to a sample if 6 of the 8 target genes showed the CIMP-like pattern. ANKRD43, FAS, HFE, LGALS3, MAL, RHOF and WWTR1 were CIMP-like when hypermethylated and DOCKS was CIMP-like when hypomethylated. Hypermethylation cut-off values were determined after screening all 20 samples.

The MethylMagnet results agreed with the MethylLight based assignments in all but one instance. The apparent CIMP⁺ sample 12-064 had been assigned the CIMP⁻ phenotype in the original validation of the pattern.

1. A method for detecting methylation of a target polynucleotide in a sample comprising:
a) cleaving a genomic DNA sample containing at least one methylated target polynucleotide with a restriction enzyme that does not cleave the methylated target polynucleotide;
b) contacting the cleaved genomic DNA with an immobilized MBD, thereby immobilizing methylated genomic DNA from the sample;
c) optionally, recovering the methylated genomic DNA from the immobilized MBD, thereby isolating methylated genomic DNA fragments;
d) contacting the methylated DNA with a primer pair that specifically hybridizes to and amplifies a target sequence of the at least one polynucleotide; and
e) detecting the amplified polynucleotide.

2. The method of claim 1, wherein the
primer pair consists of:
i) a first primer comprising a 3′ sequence complementary to a first sequence flanking the target sequence of the polynucleotide, and
ii) a second primer comprising:
(1) a 3′ sequence complementary to a second sequence flanking the target sequence of the polynucleotide, and
(2) a 5′ sequence comprising one strand of an APC.

3. The method of claim 1, wherein the detecting the amplified polynucleotide comprises
e) amplifying the target sequence from the first and second primers, wherein the amplification produces an APC;
f) transcribing at least one Abscript from the APC;
and
g) detecting the at least one Abscript transcribed in step c.

4. The method of claim 1, wherein unbound reagents and polynucleotides are washed from immobilized and captured polynucleotides following step b.

5. The method of claim 1, further comprising:
f) recovering the unbound polynucleotides comprising unmethylated DNA
g) contacting the unmethylated DNA with a primer pair that specifically hybridizes to and amplifies a target sequence of the at least one polynucleotide; and
h) detecting the amplified unmethylated polynucleotide.

6. The method of claim 1, wherein amplifying consists of performing a polymerase chain reaction.

7. The method of claim 6, wherein the polymerase chain reaction is performed with at least one of a thermostable DNA polymerase and a thermostable RNA polymerase.

8. The method of claim 1, wherein a detectably labeled nucleotide is incorporated into the at least one Abscript during step d).

9. The method of claim 8, wherein the detectably labeled nucleotide is a fluorescent nucleotide.

10. The method of claim 1, wherein detecting the at least one Abscript comprises mass spectrometry, capillary electrophoresis or thin layer chromatography.

11. The method of claim 1, wherein the at least one Abscript is 3-20 nucleotides in length.

12. The method of claim 11, wherein the at least one Abscript is 3 nucleotides in length.

13. The method of claim 3, wherein the means for directing Abscription comprises an APC.

14. The method of claim 1, wherein the at least one target polynucleotide is a methylated CpG island and the sample comprises isolated methylated genomic DNA fragments.

15. The method of claim 1, wherein the MBD is a GST-MBD2 fusion protein.

16. The method of claim 15, wherein the GST-MBD2 fusion protein is immobilized on a glutathione-containing solid support.

17. The method of claim 1, wherein the at least one polynucleotide is differentially methylated in cancer or in an imprinting related disease, disorder or syndrome.

18. The method of claim 1, wherein the genomic DNA sample is a formalin fixed, paraffin embedded sample.
